(19) United States
(12) Patent Application Publication
(10) Pub. No.: US 2009/0176202 A1
(43) Pub. Date: Jul. 9, 2009

(54) Methods of Detecting Inhibitors of VIF-Mediated APOBEC3G Degradation and HIV

(75) Inventors: Yonchu Jenkins, Oakland, CA (US); Guoping Su, Mountain View, CA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: RIGEL PHARMACEUTICALS, INC., South San Francisco, CA (US)

(21) Appl. No.: 12/054,272

(22) Filed: Mar. 24, 2008

Related U.S. Application Data
(60) Provisional application No. 60/896,759, filed on Mar. 23, 2007.

(51) Int. Cl. C12Q 1/70; C12Q 1/68; C12Q 1/66; C12N 5/00

(52) U.S. Cl. 435/5; 435/6; 435/8; 435/325

(57) ABSTRACT

The invention comprises methods and cell lines for assaying APOBEC3G degradation and methods for identifying inhibitors of APOBEC3G degradation. The invention also provides methods of identifying inhibitors of HIV infection. The methods of the invention are useful for identifying inhibitors of viral infection, in particular, the methods of the invention are useful for treating retroviral infection.

This application claims the benefit of U.S. provisional application 60/896,759, filed Mar. 23, 2007.

### BACKGROUND OF THE INVENTION

1. Field of the Invention

The present invention is in the general field of HIV therapeutics. The present invention is also in the field of targeting ubiquitin ligase pathways involved in viral replication. In particular, the present invention is directed to methods of inhibiting HIV Vif-mediated APOBEC3G degradation, inhibition of viral assembly and trafficking, and modulation of E2 function.

2. Summary of the Related Art

Eukaryotes have a wide variety of innate defenses, including antimicrobial peptides, proteolytic cascades, signaling molecules such as interferons and specialized phagocytic cells. The various defense systems work together against pathogens (Beutler, B. and Hoffmann, J., Curr. Opin. Immunol. 16, 1-3 (2004); Samuel, C. E., Clin. Microbiol. Rev. 14, 778-809 (2001)), and are poised at all times to readily neutralize invading organisms. Even the outer membrane of a cell and the epithelial-cell surfaces of multicellular organisms can be considered innate immune defenses in that they function as ever-present barriers to infection.

A novel mechanism of innate immunity is the potent cellular defense that actively blocks retroviral infection. At least two cellular proteins lie at the center of this defense mechanism: APOBEC3F and APOBEC3G (Apolipoprotein B mRNA-editing enzyme-catalytic polypeptide-like 3G). These cellular proteins function by ‘hitchhiking’ with newly produced viral particles until the viral particle encounters a new target cells. Then, during synthesis of the first retroviral DNA strand (minus strand), which is an obligate step in the retroviral life cycle, APOBEC-dependent deamination of cytosine (C) residues results in the accumulation of excessive levels of uracil (U). This pre-mutagenic lesion leads to the demise of the invading retrovirus on its replication because uracil is recognized as thymine (T) by the viral reverse transcriptase and adenine (A) is incorporated into the newly synthesized second (plus) DNA strand rather than guanine (G). This process of lesion fixation can therefore produce a detrimental level of mutations in the retroviral genome.

APOBEC3F and APOBEC3G have also been reported to possess antiviral activity independent of their catalytic function. This non-enzymatic inhibition is based on the ability of APOBEC3F/APOBEC3G to directly interfere with the process of reverse transcription and perhaps also with integration. This leads to dramatic reductions in the absolute quantity of proviral transcripts available for integration into the host genome.

APOBEC3F and APOBEC3G are closely related to another cytosine deaminase, activation-induced deaminase (AID), which also uses C to U deamination to initiate three distinct types of immunoglobulin-gene diversification: somatic hypermutation, gene conversion and class-switch recombination. These processes are an integral part of the DNA-level modifications that drive maturation of the vertebrate antibody response to pathogens. The apparent deliberate use of DNA deamination by APOBEC3F, APOBEC3G and AID therefore constitutes a striking mechanistic parallel between innate and adaptive immunity, both of which use deamination to restrict infection.

The virion infectivity factor (Vif) of HIV mediates APOBEC3G degradation. Vif forms a complex with APOBEC3G and enhances APOBEC3G ubiquitination and proteasome targeting resulting in reduced steady-state APOBEC3G levels and decreased in protein half-life. The ubiquitination of APOBEC3G leads to its degradation and thereby prevents APOBEC3G from being incorporated into assembling virus particles. The functional interaction between Vif and the APOBEC3G proteins is likely to be delicately balanced such that even minor disturbances could influence the outcome of an infection. Vif directly binds APOBEC3G and it also binds to the cullin5/elonginC component of a ubiquitin ligase complex composed of cullin5/elonginB/elonginC/rbx 1. Recruitment of APOBEC3G to this complex results in polyubiquitination of APOBEC3G. The polyubiquitinated APOBEC3G protein becomes a substrate for the 26S proteasome and is rapidly degraded. The overall effect is a dramatic reduction in steady state APOBEC3G levels, which prevents APOBEC3G from getting packaged into assembling virus particles.

It might even be possible to take advantage of this balance therapeutically. One possible strategy is to use Vif-binding compounds that are able to directly prevent Vif from functioning (Harris and Liddament, Nature Reviews 4, 868-877 (2004)). This would presumably leave the cellular APOBEC proteins free to restrict infection. However, similar to anti-HIV-1 therapies that are directed towards viral reverse transcriptases or proteases, this approach might eventually succumb to viral ‘escape’ mutants. The intrinsically high level of genetic variation in a retroviral population (even without APOBECs) would probably undermine this approach by creating Vif variants that would no longer be bound by the inhibitors. However, in combination with other anti-retroviral drugs, such a compound would fortify the pharmaceutical anti-HIV-1 arsenal and further reduce the possibility of viral relapse.

It has been argued that retroviruses such as HIV-1 are on the edge of a genetic abyss, with a mutation load so high that, if pushed higher, it might drive the virus to extinction (Loeb, L. A. et al., Proc. Natl. Acad. Sci. USA 96, 1492-1497 (1999)). Retroviral hypermutation by APOBEC3G results in 10- to 1000-fold increase in the viral mutation load in model cell-culture systems, showing that viral nucleic acid can be made genetically inert through APOBEC-dependent deamination (Harris and Liddament, Nature Reviews 4, 868-877 (2004)). Thus, a second approach is to use a ‘molecular shield’ in vivo, that is, a compound that would protect APOBECs from Vif but not interfere with APOBEC antiretroviral activities (Harris and Liddament, Nature Reviews 4, 868-877 (2004)). This approach might be advantageous over Vif inhibitors because APOBEC is a comparatively stable cellular target.

### BRIEF SUMMARY OF THE INVENTION

The present invention provides methods and cell lines for assaying APOBEC3G degradation and detecting inhibitors of APOBEC3G degradation. The methods and cell lines are useful for identifying inhibitors of HIV replication and infection.

In a first aspect, the invention provides methods for assaying APOBEC3G degradation.

In a second aspect, the invention provides cell lines for assaying APOBEC3G degradation.

In a third aspect, the invention provides methods for detecting inhibitors of APOBEC3G degradation.

The foregoing only summarizes certain aspects of the invention and is not intended to be limiting in nature. These aspects and other aspects and embodiments are described more fully below. All patent applications and publications of any sort referred to in this specification are hereby incorporated by reference in their entirety. In the event of a discrepancy between the express disclosure of this specification and a patent application or publication incorporated by reference, the express disclosure of this specification shall control.

### BRIEF DESCRIPTION OF THE DRAWINGS

FIG. 1 shows ubiquitination in the life cycle of HIV.

FIG. 2 shows current therapeutic targets in the life cycle of HIV.

FIG. 3 shows that targeting Vif function could result in increased antiviral activity of APOBEC3G.

FIG. 4 shows Vif-mediated degradation of APOBEC3G by mediation of ubiquitination of APOBEC3G, thereby preventing APOBEC3G from being incorporated into assembling virus particles.

FIG. 5 shows inhibition of Vif-mediated degradation of APOBEC3G, permitting incorporation of APOBEC3G into assembling virions.

FIG. 6 shows antiviral effects resulting from APOBEC3G-mediated deamination of the HIV genome.

FIG. 7 shows a schematic of Example 1, a cell-based assay for compounds that inhibit Vif-mediated APOBEC3G degradation.

FIG. 8 shows an APOBEC3G-specific HIV single-cycle infectivity assay, which measures cellular APOBEC3G stabilization, compound antiviral effect, and APOBEC3G specificity of inhibition.

FIG. 9 shows the characterization of targets of inhibitors of APOBEC3G degradation.

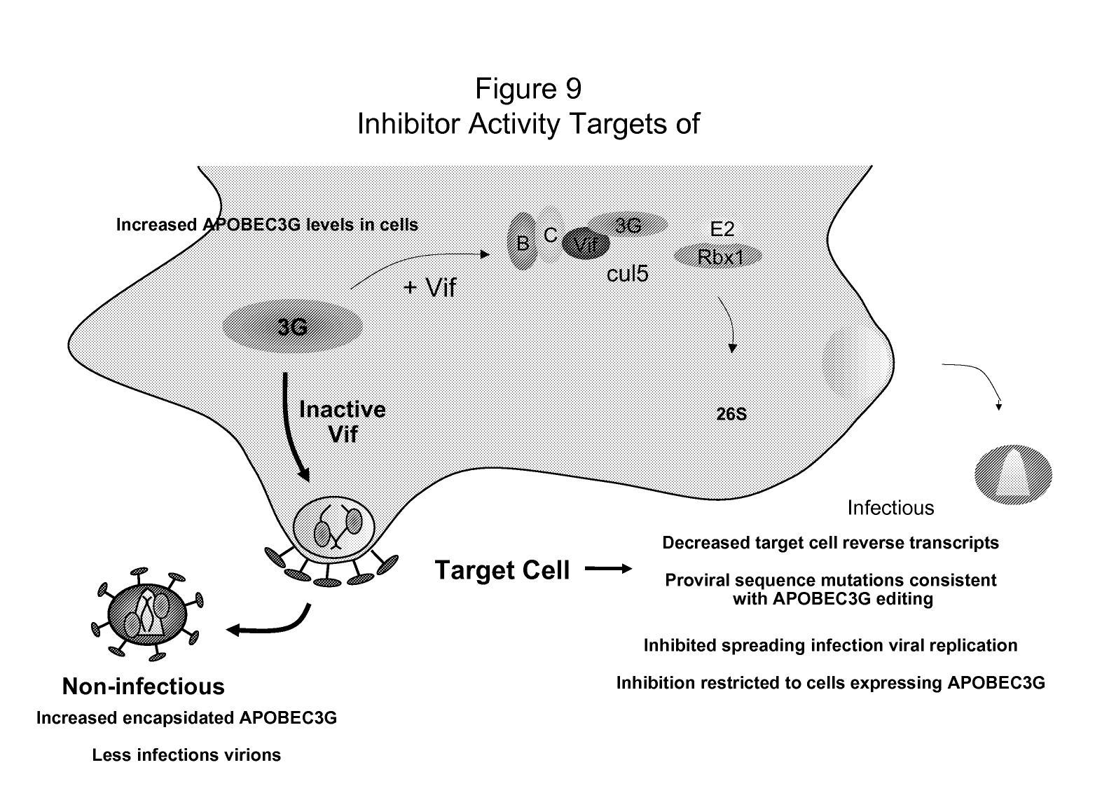

FIG. 10 shows a schematic for HIV virion particle isolation by sedimentation through sucrose (example 9).

FIG. 11 shows the increase in APOBEC3G in virions using several utilizing 293T cells and T cells.

FIG. 12 shows a schematic for a single cycle replication assay for measuring HIV infectivity (example 4).

FIG. 13 shows the dose dependent HIV inhibitory activity of some compounds identified by the methods of the invention.

FIG. 14 shows a schematic for HIV cDNA quantitation and sequence analysis (example 12 and 13).

FIG. 15 shows the decreased HIV cDNA levels observed in T cells infected with compound-treated viruses.

FIG. 16 shows mutations caused by APOBEC3G deamination in viral DNAs from cells infected with viruses produced in the presence of some compounds identified by the methods of the invention.

FIG. 17 shows the experimental scheme for viral replication analysis of Example 12.

FIG. 18 shows the HIV replication inhibitory activity of a compound identified by the methods of the invention.

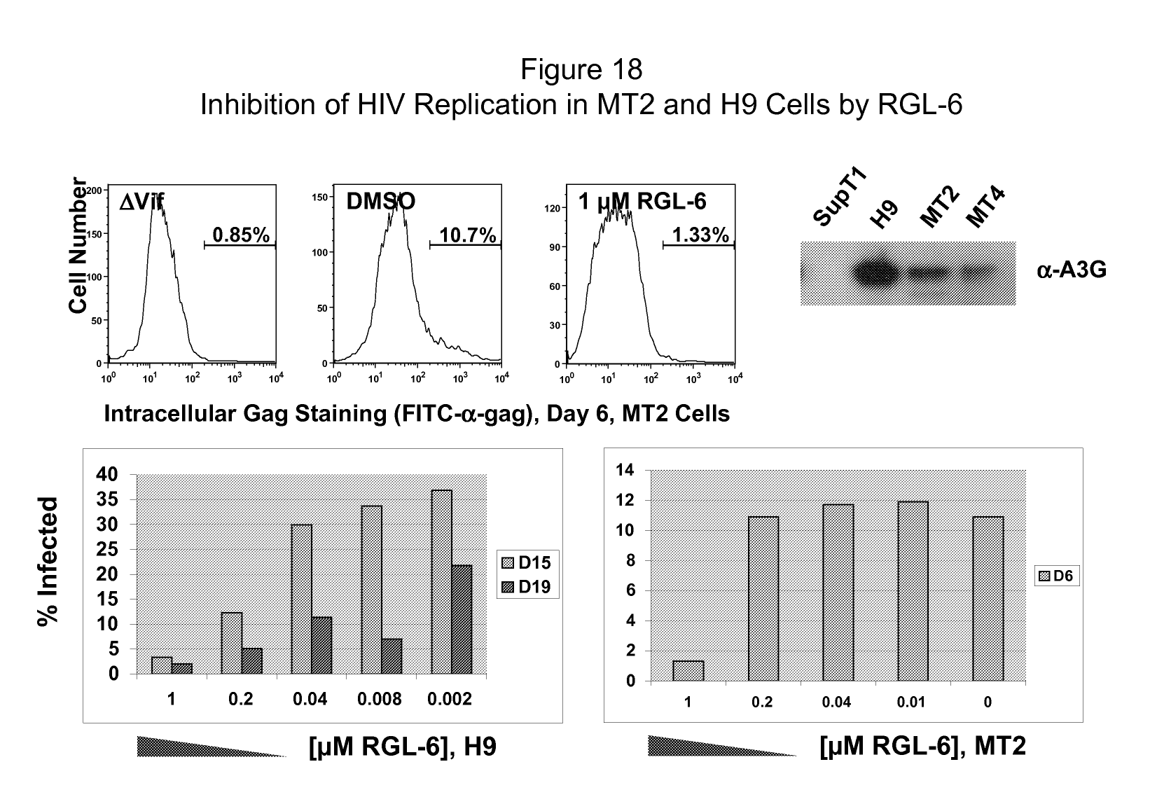

FIG. 19 shows that the HIV inhibitory activity of the compound in FIG. 18 is specific for cells that express APOBEC3G.

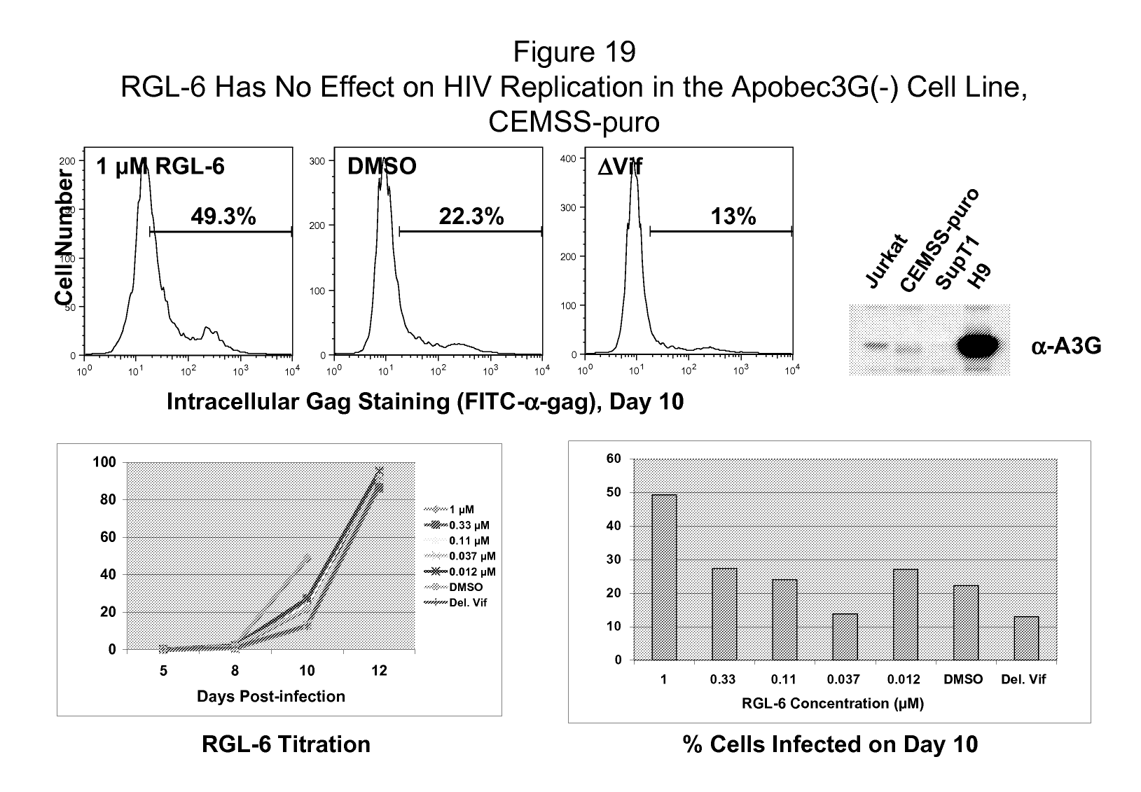

FIG. 20 shows the dose dependent increase of APOBEC3G levels in virions upon cell treatment with some compounds.

FIG. 21 shows the correlation between detection of virion-incorporated APOBEC3G by western blotting compared to luciferase activity measurement.

FIG. 22 shows the correlation between increasing levels of supernatant HIV capsid protein and increasing levels of APOBEC3G-luciferase activity, Example 7.

FIG. 23 shows the analysis method of measuring inhibition of Example 4.

### DETAILED DESCRIPTION OF THE PREFERRED EMBODIMENTS

The invention provides methods and cell lines for assaying APOBEC3G degradation and methods for identifying inhibitors of APOBEC3G degradation. The invention also provides methods of identifying inhibitors of HIV infection.

In the first aspect, the invention provides methods for assaying for APOBEC3G in an HIV sample. In one embodiment, the invention provides a method of assaying APOBEC3G derived from HIV comprising: a) providing a cell comprising at least one reporter gene, wherein expression of the at least one reporter gene is induced by an HIV specific promoter, wherein the cell further comprises at least one type of HIV receptor; b) contacting the cell with HIV under conditions that allow entry of HIV into the cell; and c) measuring a signal generated by the reporter gene; wherein the magnitude of the signal is inversely proportional to the amount of APOBEC3G in the HIV. In one embodiment, the cell is HeLa line. The HIV specific promoter is induced by HIV nucleic acids, proteins or fragments thereof. In another embodiment, the cell comprises two reporter genes. In still another embodiment, the reporter genes are genes for luciferase and/or 0-galactosidase. In yet another embodiment, the cell comprises HIV receptors CD4 and CCR5.

In the method of the first aspect, the degree of expression of the reporter gene is inversely related to the level of ABOBEC3G in the HIV used for contacting the cell in step b). HIV particles with high levels of APOBEC3G do not induce the expression of the reporter gene because no HIV nucleic acid or protein is produce. APOBEC3G promotes high levels of mutation in the HIV genome by converting cytosine to uracil. This hypermutated HIV genome may be targeted for degradation before integration in the host DNA, or if integrated and expressed, the encoded proteins may also be defective and not function as inducers of the HIV specific promoter.

“Reporter genes” as recited herein code for a “reporter molecule” that possesses or is capable of generating or inducing, directly or indirectly, a detectable signal.

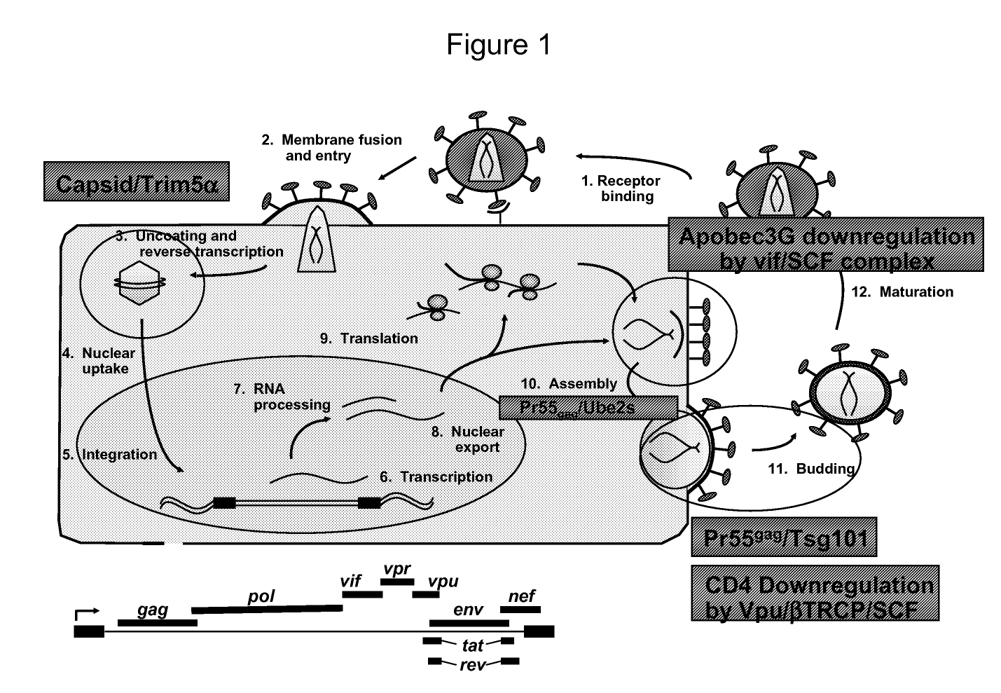

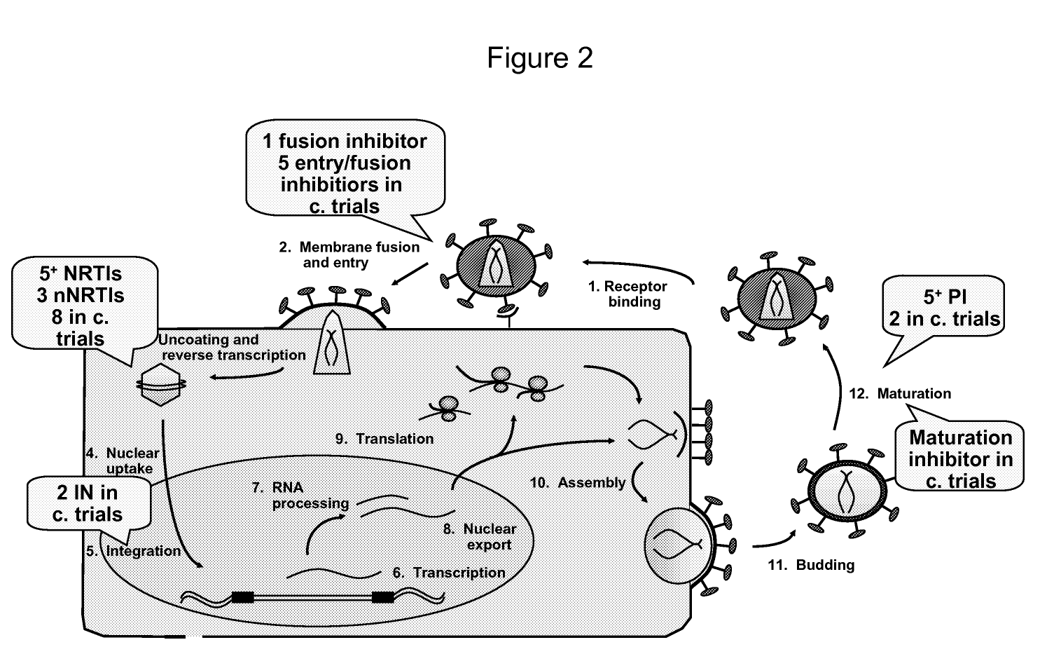

The life cycle of HIV shown in FIGS. 1 and 2 comprise a number of important processes. (In FIG. 2, NRTI: nucleoside and nucleotide analog reverse transcriptase inhibitor, nNRTI: non-nucleoside reverse transcriptase inhibitor, PI: protease inhibitor, IN: integrase inhibitor.) The size of the HIV particle is around 100-120 nm in diameter. A viral membrane encloses an inner particle core. The glycoprotein gp160 is embedded in the viral membrane and is involved in binding and membrane fusion with target cells. The inner core contains the viral RNA and some enzymes such as reverse transcriptase, protease, and integrase. The viral RNA encodes a number of proteins as shown in FIG. 1. The encoded proteins include the structural polyprotein Gag; Pol, which is processed into a protease, integrase and reverse transcriptase; and Env, which is further post-translationally modified to produce the viral glycoprotein. The viral RNA also codes for a number of smaller proteins such as Vif, Vpr, Vpu, Tat, Rev, and Nef. These smaller proteins play important regulatory functions to ensure replication of HIV and production of infectious particles.

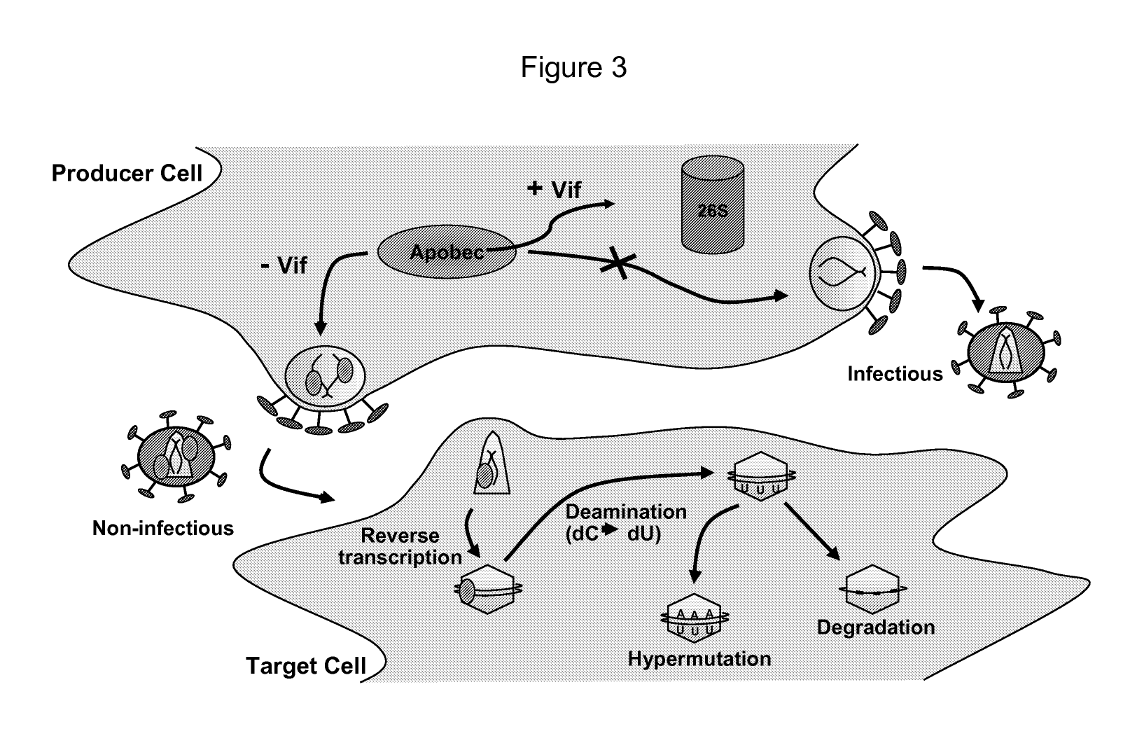

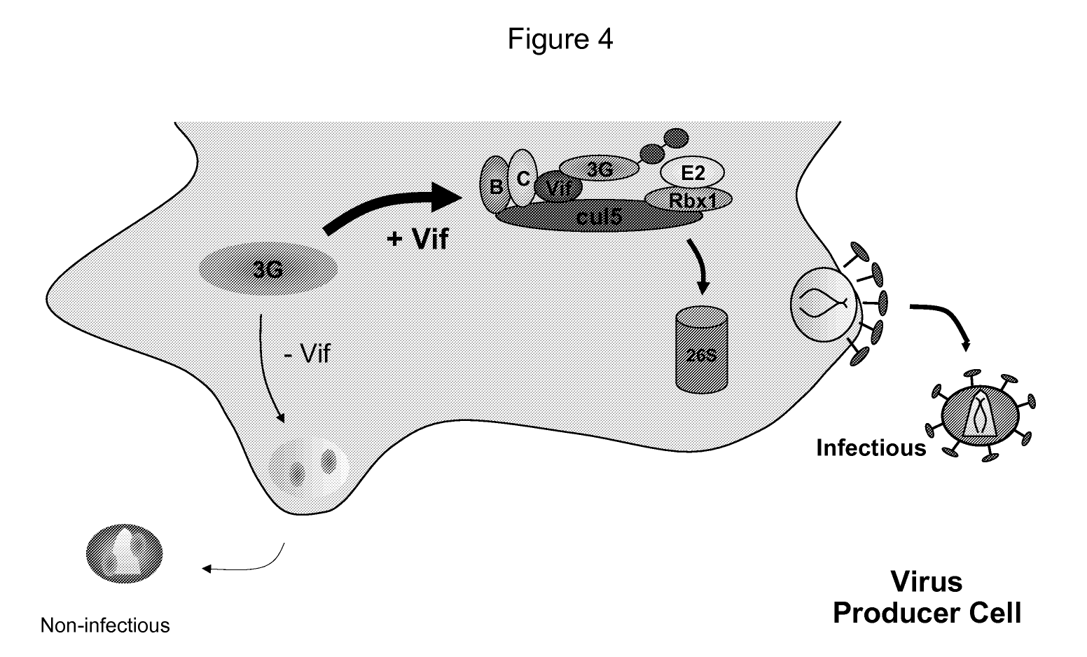

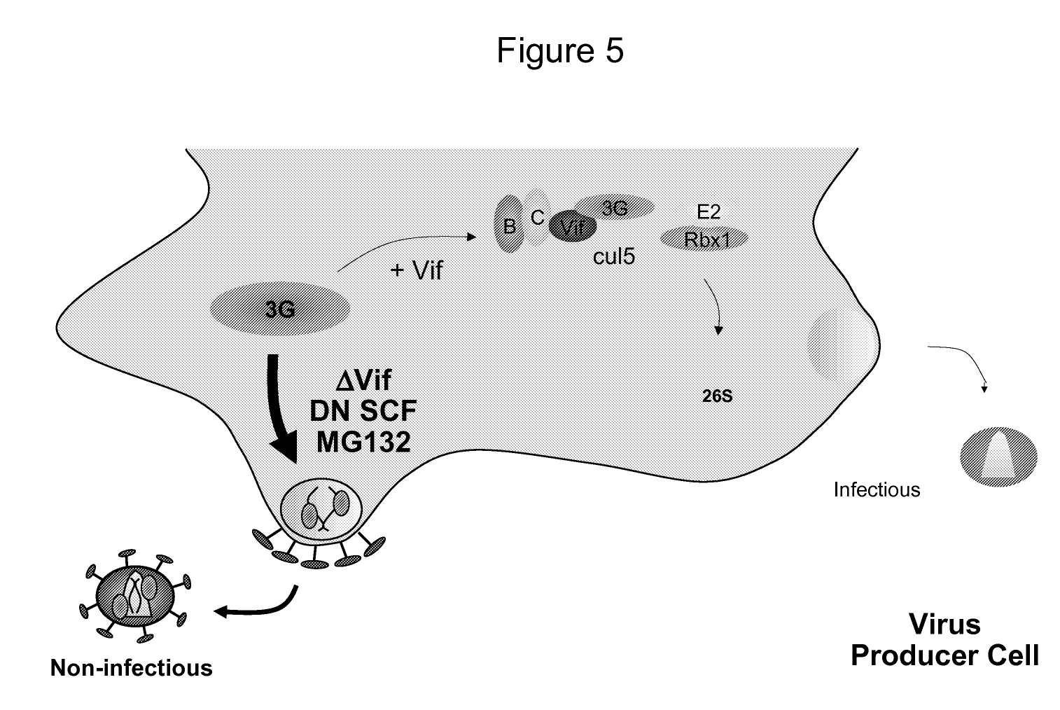

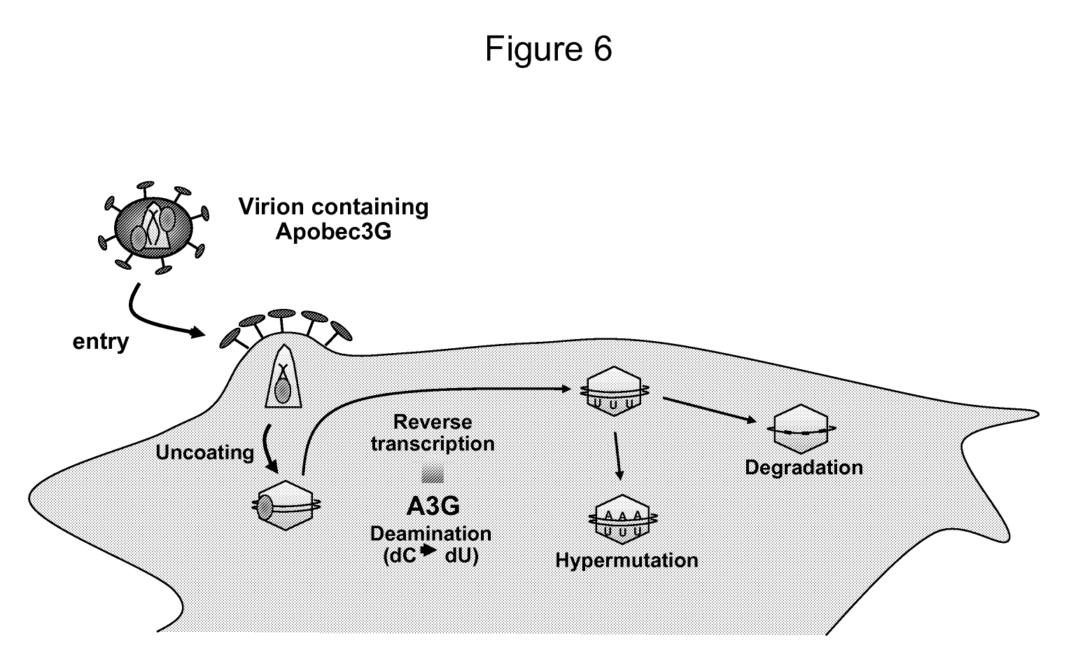

In cells infected with HIV, Vif acts during assembly of HIV virions to reduce the cellular levels of the host protein APOBEC3G by promoting polyubiquitination of APOBEC3G and subsequent degradation by the 26S proteasome (FIG. 3-5). The mature HIV particles assembled from infected cells do not incorporate APOBEC3G. In infected cells treated with inhibitors of APOBEC3G degradation, the HIV particles do incorporate APOBEC3G even with a functional Vif. HIV particles with APOBEC3G fail to establish a productive infection in target cells. APOBEC3G interferes with reverse transcription and perhaps integration to reduce the amount of proviral transcripts available for integration. Additionally, APOBEC3G promotes deamination of cytosine (C) during the synthesis of the first retroviral DNA strand (minus strand) and the accumulation of excessive levels of uracil (U) in the DNA strand (FIG. 6). APOBEC3G may also promote deamination of cytosine (C) in the viral RNA. Thus, the end effect of APOBEC3G induced deamination is a transition mutation from C/G to T/A base pairing that affects virus viability. The uracil in the hypermutated DNA minus strand is recognized as thymine (T) by the viral reverse transcriptase and adenine (A) is incorporated into the newly synthesized DNA plus strand rather than guanine (G). This pre-mutagenic lesion in the DNA leads to the demise of the invading retrovirus because subsequent integration and replication may result in no production of viral proteins or defective viral proteins. In addition, DNA containing uracil may also be targeted for degradation even before integration into the host DNA.

The method of the first aspect of the invention is useful for assaying APOBEC3G in HIV particles by determining whether HIV particles produced from cells treated or not treated with inhibitors of APOBEC3G degradation are infectious or support production of infectious HIV particles. Accordingly, in the method of the first aspect the HIV used is produced from cells treated with an inhibitor (or putative inhibitor) of APOBEC3G degradation.

In one embodiment, the expression of the reporter gene is driven or induced by an HIV specific promoter. The HIV specific promoter may be activated by any HIV nucleic acid, protein or fragments thereof. For example, the reporter gene may be coupled to a promoter region that binds to an HIV protein thereby activating the expression of the reporter gene. The HIV protein serves as a promoter inducer or transcription factor. Any of the proteins encoded by the HIV genome can serve as a promoter inducer or transcription factor. In one embodiment, the promoter region may be a region that binds to the HIV Tat protein. In another embodiment, the promoter region is HIV LTR that contains a promoter region that binds to Tat.

According to the method of the first aspect of the invention, HIV particles that incorporated APOBEC3G would produce defective reverse transcribed DNA that may be degraded before integration into the host DNA. As a result, the HIV specific promoter is not induced because no HIV specific protein or nucleic acid is produced, and there is no expression of the reporter gene. Alternatively, the defective HIV DNA may still be integrated into the host DNA but defective proteins are produced. The defective proteins may either be fragments due to premature stop codons or full-length mutated proteins. The fragments or mutated proteins would not serve as transcription factors for the expression of the reporter genes because they may bind very poorly to the promoter region. The fragments or mutated proteins may also be degraded and unavailable to bind to the promoter region.

The method of the first aspect of the invention is also useful to detect defective APOBEC3G, or HIV that did not incorporate APOBEC3G. Such particles would support the production of HIV proteins that would be available to function as inducers for the expression of the reporter gene.

In the second aspect, the invention provides cell lines for assaying APOBEC3G degradation. In one embodiment, the invention provides a cell comprising genes that code for Vif and an APOBEC3G fusion protein, wherein the gene that codes for the APOBEC3G fusion protein comprises a nucleic acid sequence encoding APOBEC3G and a reporter gene. In one embodiment, the reporter gene codes for firefly luciferase, or green fluorescent protein and variants from aquoria and renilla. In still another embodiment, the product of the gene that codes for APOBEC3G fusion protein is an APOBEC3G protein fused at the C-terminus to firefly luciferase. In yet another embodiment, the cell is a HeLa cell. In another embodiment, the cell is a T-cell stably expressing an APOBEC3G fusion protein. Routine methods known to those skilled in the art for making such fusion genes and transfecting cells to express them can be used.

In the third aspect, the invention provides methods for identifying inhibitors of APOBEC3G degradation. In a first embodiment of the third aspect, the invention provides a method of identifying inhibitors of HIV infectivity comprising:
a) producing HIV virus particles in two cultures of virus producing cells, wherein the HIV virus particles are produced in a first culture in the presence of a test compound and in a second culture in the absence of the test compound;
b) measuring the amount of APOBEC3G in the HIV virus particles produced by the virus producing cells; and
c) comparing the amount of APOBEC3G in the HIV virus particles produced in the virus producing cells in the presence of the test compound to the amount of APOBEC3G in HIV virus particles produced in the virus producing cells in the absence of the test compound,
wherein a greater amount of APOBEC3G in the HIV virus particles produced from the virus producing cells in the presence of the test compound indicates that the test compound is an inhibitor of HIV infectivity.

In one embodiment of the first embodiment, the virus producer cells are 293T cells. In some embodiments of the first embodiment, the virus producer cells are T lymphocytes that express either endogenous APOBEC3G or exogenous APOBEC3G. A T cell that endogenously expresses APOBEC3G is H9. A T cell that exogenously expresses APOBEC3G is CEMSS-A3G that has been transduced with a retroviral vector expressing APOBEC3G.

In a second embodiment of the third aspect, the invention provides a method of identifying an inhibitor of vif-mediated APOBEC3G degradation, comprising:
a) producing HIV virus particles in first and second virus producing cell cultures, wherein the HIV virus particles are produced in the first cell culture in the presence of a test compound and in the second test culture in the absence of the test culture, wherein the virus producing cells of the first and second cell cultures contain APOBEC3G;
b) producing HIV virus particles in third and fourth virus producing cell cultures, wherein the HIV virus particles are produced in the third cell culture in the presence of a test compound and in the fourth cell culture in the absence of the test compound, wherein the third and fourth virus producing cell cultures do not contain APOBEC3G;
c) measuring the infectivity of the HIV virus particles produced in the first, second, third, and fourth virus producing cell cultures;
d) comparing the infectivity of the HIV virus particles produced in the first cell in the first cell culture to the infectivity of the HIV virus particles produced in the second cell culture; and
e) comparing the infectivity of the HIV virus particles produced in the third cell culture to the infectivity of the HIV virus particles produced in the fourth cell culture,
wherein a reduction of infectivity of the HIV virus particles produced in the first cell culture compared to the second cell culture and no reduction in infectivity of the HIV virus particles produced in the third cell culture compared to the fourth cell culture indicates that the test compound is an inhibitor of vif-mediated APOBEC3G degradation.

In one embodiment of the second embodiment, the virus producer cells are 293T cells. In some embodiments of the second embodiment, the virus producer cells are T lymphocytes that express either endogenous APOBEC3G or exogenous APOBEC3G.

HIV infectivity refers to the ability of HIV to produce infected cells or ability to propagate more virus in other cells.

In a third embodiment of the third aspect, the invention provides a method of identifying an inhibitor of HIV infectivity comprising:
a) producing HIV virus particles in a first cell culture and a second cell culture, wherein the HIV virus particles are produced in the first cell culture in the presence of a test compound and in the second cell culture in the absence of the test compound;
b) infecting a third and fourth cell cultures with the HIV virus particles produced in the first and second cell cultures, respectively;
c) culturing the third and fourth cell cultures;
d) measuring the level of HIV nucleic acid in the third and fourth cell cultures; and
e) comparing the amount of HIV nucleic acid in the third cell culture to the amount of HIV nucleic acid in the fourth cell culture,
wherein a lesser amount of HIV nucleic acid in the third cell culture compared to the fourth cell culture indicates that the test compound is an inhibitor of HIV infectivity.

In one embodiment of the second embodiment, the virus producer cells are 293T cells. In some embodiments of the second embodiment, the virus producer cells are T lymphocytes that express either endogenous APOBEC3G or exogenous APOBEC3G.

In a fourth embodiment of the third aspect, the invention provides a method of identifying an inhibitor of HIV infectivity comprising:
a) producing HIV virus particles in a first cell culture and second cell culture, wherein the HIV virus particles are produced in the first cell culture in the presence of a test compound and in the second test culture in the absence of the test compound;
b) infecting a third cell culture and a fourth cell culture with the virus produced in the first cell culture and second cell culture, respectively;
c) culturing the third and fourth cell cultures;
d) measuring the amount of mutations in HIV nucleic acid in the third and fourth cell cultures; and
e) comparing the amount of mutations in HIV nucleic acid in the third cell culture to the amount of mutations in HIV nucleic acid the fourth cell culture,
wherein a greater amount of mutations in HIV nucleic acid in the third cell culture compared to the fourth cell culture indicates that the test compound is an inhibitor of HIV infectivity.

In one embodiment of the fourth embodiment, the virus producer cells are 293T cells. In some embodiments of the third embodiment, the virus producer cells are T lymphocytes that express either endogenous APOBEC3G or exogenous APOBEC3G.

In a fifth embodiment of the third aspect, the invention provides a method of identifying inhibitors of Vif-mediated APOBEC3G degradation comprising: a) providing a cell that expresses Vif and APOBEC3G fusion protein, wherein the APOBEC3G fusion protein comprises APOBEC3G fused to a reporter molecule; b) contacting the cells with a compound to be tested under conditions that allow entry of the compound into the cell; and c) identifying the signal of the reporter molecule; wherein the magnitude of the signal is proportional to the inhibitory activity of the compound. In some embodiments of the fifth embodiment, the cells are HeLa cells. In some embodiments of the fifth embodiment, the cells are T lymphocyte cells. In the fifth embodiment, the reporter molecule can be luciferase.

In a sixth embodiment of the third aspect, the invention provides a method of identifying inhibitors of Vif-mediated APOBEC3G degradation comprising:
a) providing cells that express Vif (or HIV) and an APOBEC3G fusion protein, wherein the APOBEC3G fusion protein comprises APOBEC3G fused to a reporter molecule;
b) contacting the cells with a compound to be tested under conditions that allow entry of the compound into the cells;
c) and measuring the signal of the reporter molecule in the cell lysates;
wherein the magnitude of the signal is proportional to the inhibitory activity of the compound.

In one embodiment of the sixth embodiment, the cells are 293T cells. In some embodiments of the sixth embodiment, the virus production cells are T lymphocyte cells. In some embodiments of the sixth embodiment, the virus production cells are H9 cells. In other embodiments of the sixth embodiment, the virus production cells are CEMSS-A3G cells.

In some embodiments of the sixth embodiment, the reporter molecule is luciferase. In other embodiments, the reporter molecule is β-galactosidase. In all embodiments, the reporter activity can be normalized relative to the total cellular protein.

In a seventh embodiment of the third aspect, the invention provides a method of identifying inhibitors of Vif-mediated APOBEC3G degradation comprising:
a) providing cells that express HIV and APOBEC3G fusion protein, wherein the APOBEC3G fusion protein comprises APOBEC3G fused to a reporter molecule;
b) contacting the cells with a compound to be tested under conditions that allow entry of the compound into the cell;
c) and measuring the signal of the reporter molecule in the cell supernatant;
wherein the magnitude of the signal is proportional to the inhibitory activity of the compound.

In one embodiment of the seventh embodiment, the cells are 293T cells. In another embodiment of the seventh embodiment, the virus production cells are T lymphocyte cells. In some embodiments of the seventh embodiment, the virus production cells are H9 cells. In still another embodiment of the seventh embodiment, the virus production cells are CEMSS-A3G cells.

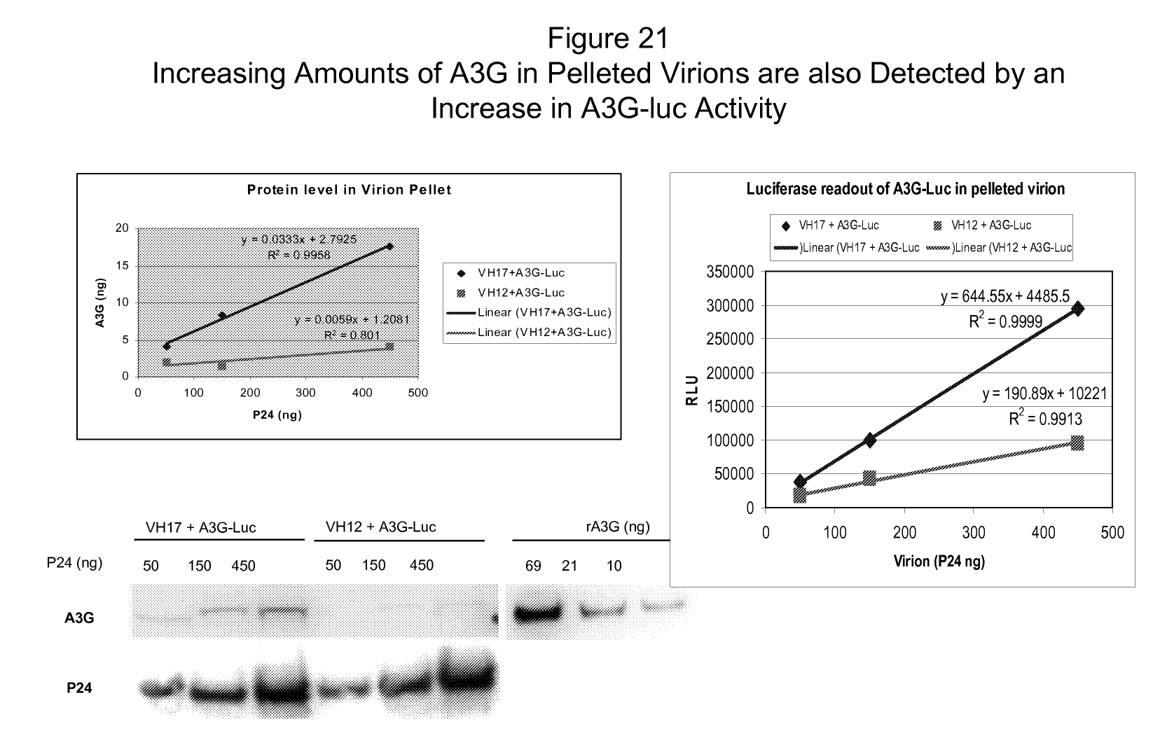

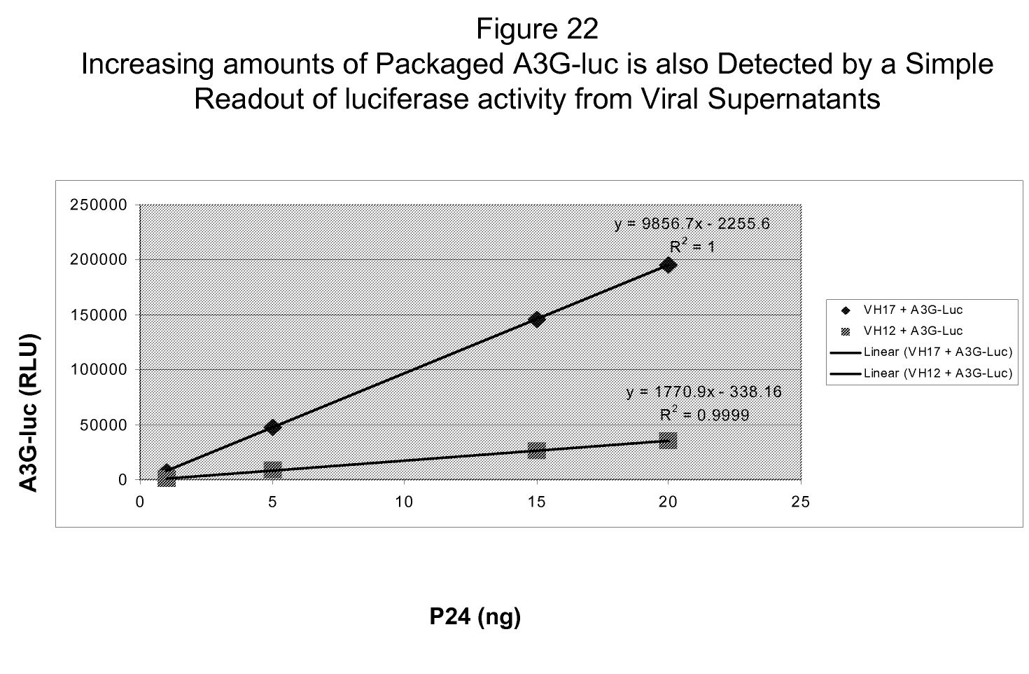

In some embodiments, the reporter molecule is luciferase. FIG. 21 shows an increased amount of APOBEC3G-luciferase in pelleted virions as detected by luciferase activity for cells treated with wild type vif and delta vif. Similarly, FIG. 22 shows an increased amount of packaged APOBEC3G-luciferase detected by luciferase activity from viral supernatants for cells treated with wild type vif and delta vif. In other embodiments, the reporter molecule is β-galactosidase. In all embodiments, the reporter activity can be normalized relative to the total cellular protein.

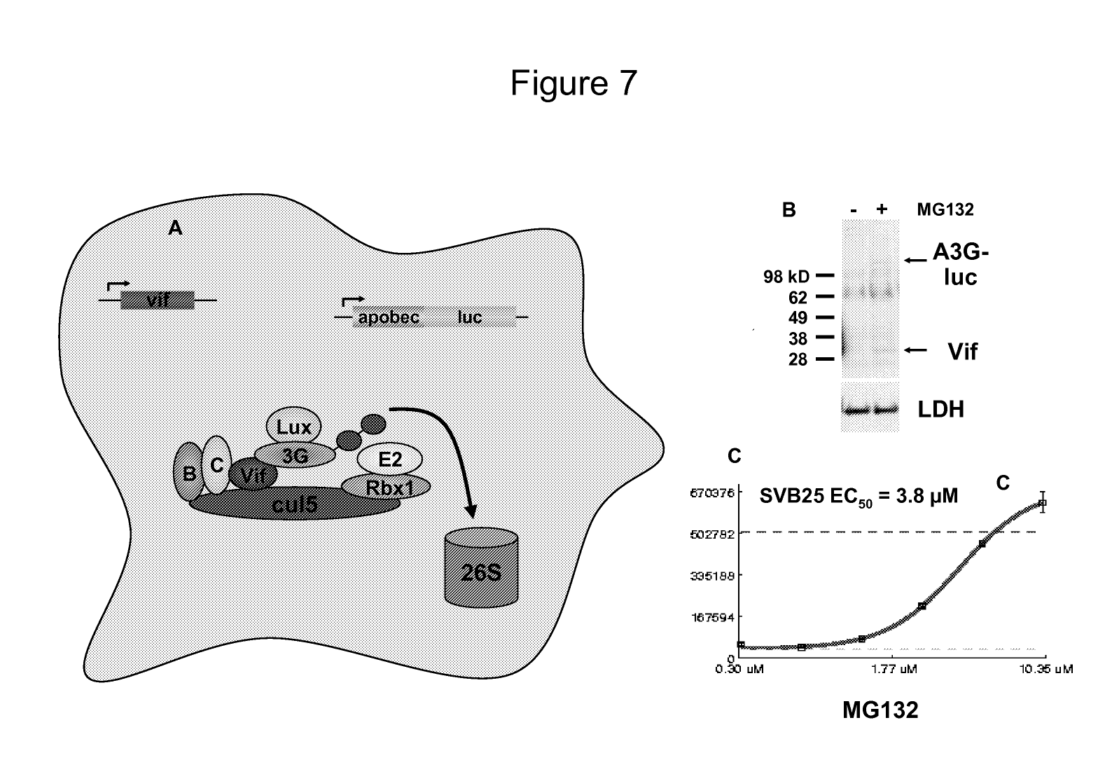

FIG. 7 shows a schematic for the method according to the third aspect of the invention. Cells contacted with a compound that is an inhibitor of APOBEC3G degradation would produce a signal from the reporter molecule even in the presence of Vif. Vif may still function properly and bind to the APOBEC3G fusion protein, but the inhibitor compound prevents the ubiquitination of the APOBEC3G fusion protein and its subsequent degradation. If the compound is incapable of preventing the degradation of the APOBEC3G fusion protein, there would be no signal because the APOBEC3G fusion protein would be ubiquitinated and targeted to degradation. The intensity of the signal would be proportional to the inhibitory activity of the compound.

In an eighth embodiment according to the third aspect, the invention provides a method of identifying inhibitors of APOBEC3G degradation comprising:
a) providing a first cell, wherein the first cells are HIV producer cells;
b) co-transfecting the first cells with HIV and APOBEC3G;
c) contacting the transfected first cells with a compound to be tested under conditions that allow entry of the compound into the cells;
d) harvesting HIV produced by the first cells after contacting the first cells with the compound;
e) providing second cells comprising at least one reporter gene, wherein expression of the at least one reporter gene is driven by an HIV specific promoter, wherein the second cells comprises at least one type of HIV receptor;
f) contacting the second cells with HIV harvested from d) under conditions that allow entry of HIV into the cells; and
g) measuring the signal; wherein the magnitude of the signal is inversely proportional to the inhibitory activity of the compound.

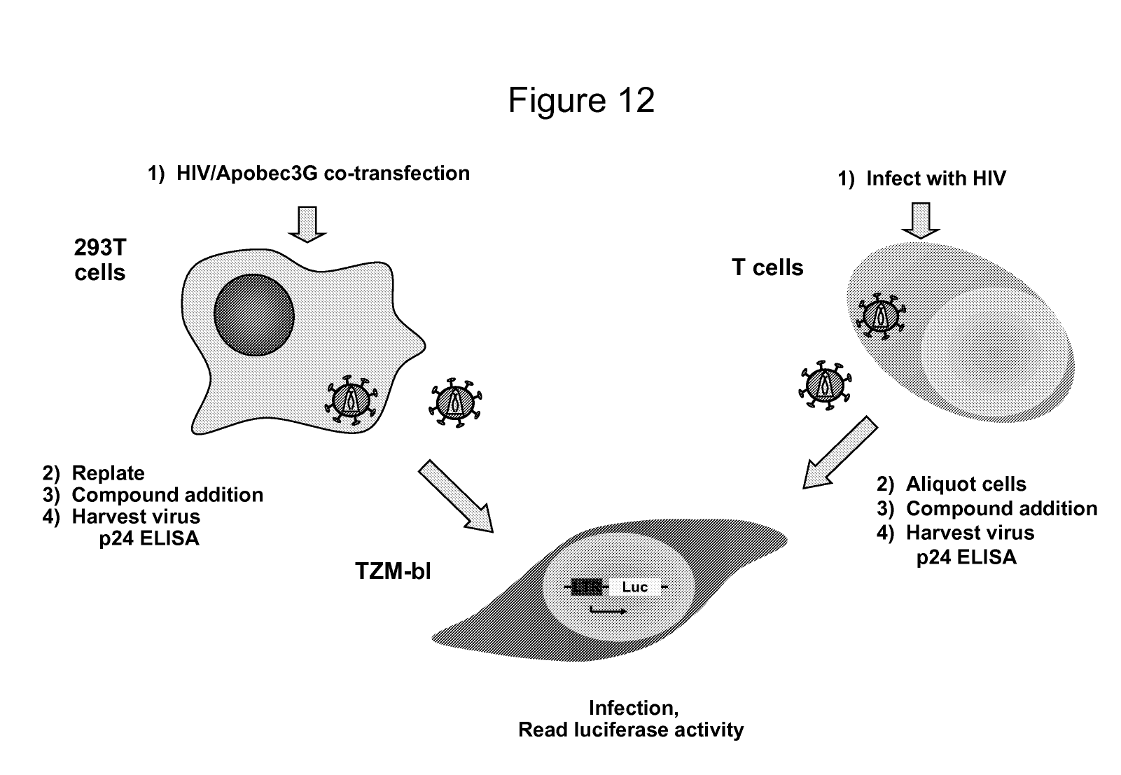

In one embodiment of the eighth embodiment of the third aspect, the first cells are 293T cells and second cells are HeLa cells. In some embodiments of the eighth embodiment, the second cell comprises two reporter genes. In some embodiments of the eighth embodiment, the second cell comprises HIV receptors CD4 and CCR5. In some embodiments of the eighth embodiment, the first cells are T lymphocyte cells. FIG. 12 illustrates one embodiment according to the third aspect of the invention.

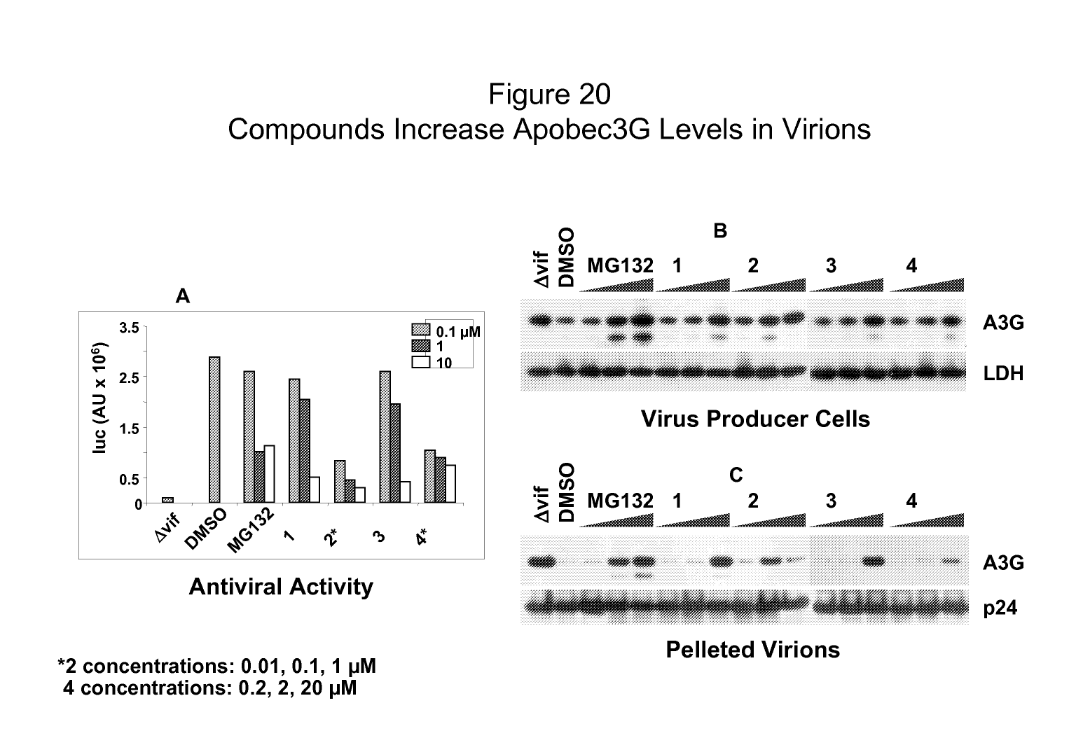

FIG. 20 shows a comparison of 293T cells and T-cells for the production of virions for assays.

In another embodiment, the invention provides a method of identifying inhibitors of Vif-mediated APOBEC3G degradation comprising:
a) providing cells that express Vif and an APOBEC3G fusion protein, wherein the APOBEC3G fusion protein comprises APOBEC3G fused to a reporter molecule;
b) infecting the T lymphocyte cells with the HIV virus,
c) after partial infectivity, contacting the cells with a compound to be tested under conditions that allow entry of the compound into the cell;
d) measuring the signal from the reporter molecule; wherein
   i) the reporter signal is measured using the cell lysate;
   ii) the reporter signal is measured using the supernatant; or
e) measuring the level of APOBEC3G protein encapsidated in virions or in lysate using western blot, or p24 ELISA; or
f) measuring the infectivity of the virions produced using TZM cell assays as described herein.

In some embodiments, the cells are 293T cells. In other embodiments, the cells are T lymphocyte cells stably expressing an APOBEC3G fusion protein.

In some embodiments, the reporter molecule is luciferase or β-galactosidase.

A preferred embodiment is measuring the reporter molecule luciferase signal and using the supernatant.

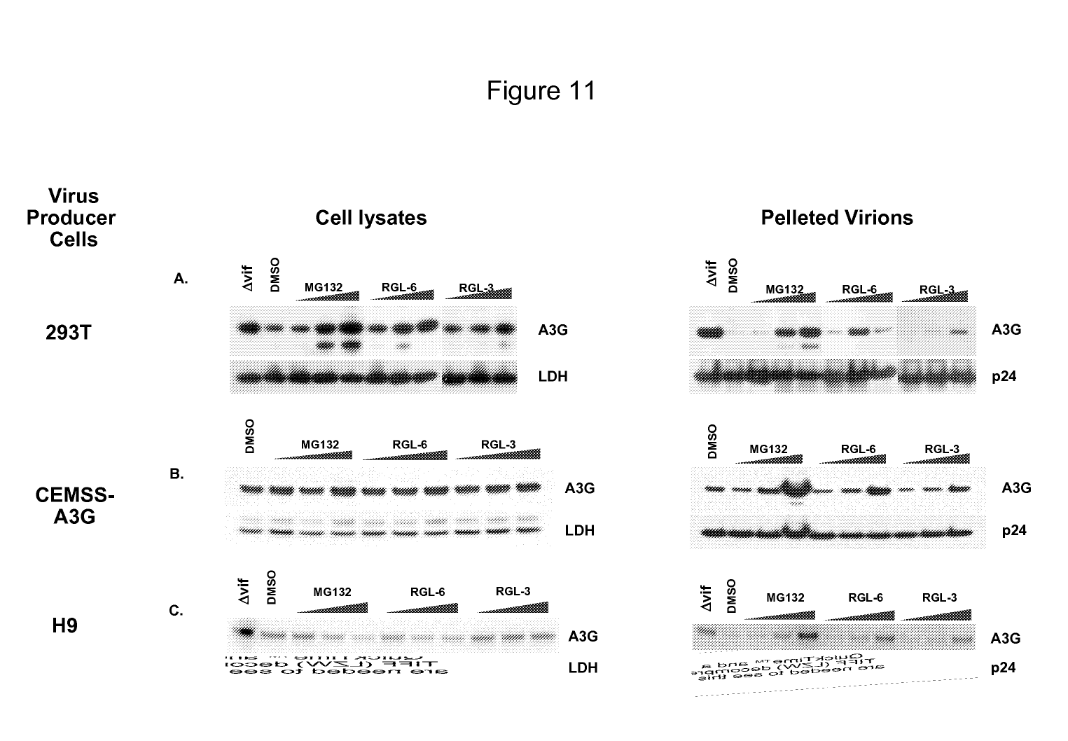

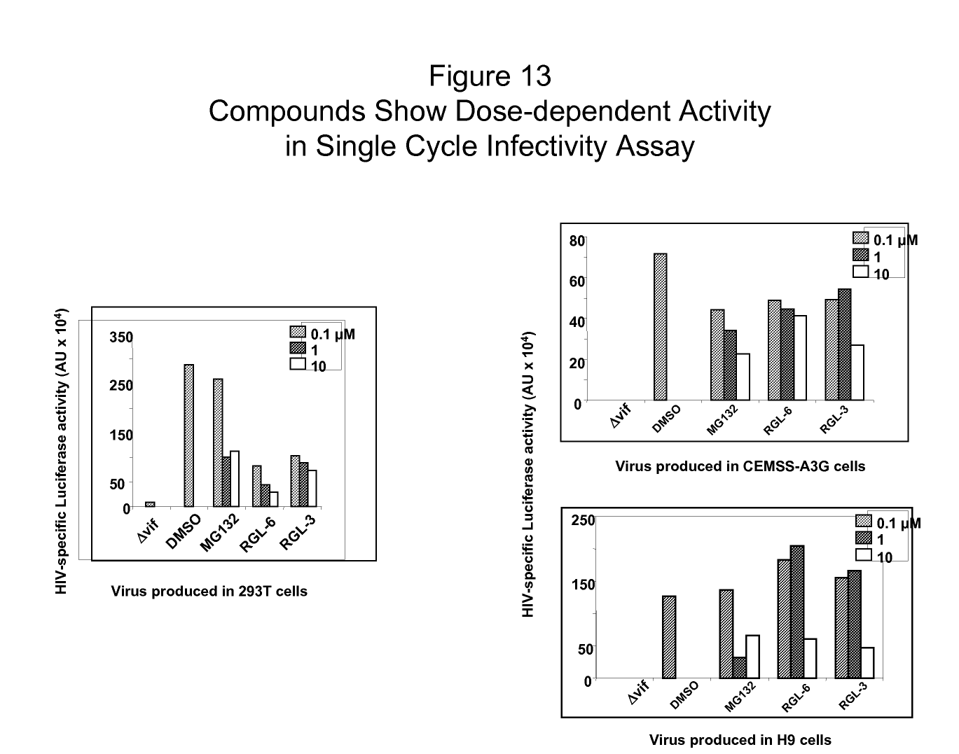

In all embodiments of the third aspect, the virus may be grown in T lymphocyte cells. HIV infects cells of the immune system and the central nervous system. T helper lymphocyte cell-lines express CD4, CCR5 or CXCR4 receptors on the cell surface, thus using T-cells for virus production cells is physiologically relevant for compound characterization in biological assays. Viruses produced from infected T cells treated with APOBEC3G degradation inhibitory compounds incorporate more APOBEC3G protein than viruses from T cells with no exposure to the inhibitory compounds, FIG. 11, and have lower infectivity by luciferase activity, FIG. 13.

We have found that the APOBEC3G-fusion assay provides several advantages. In comparison to ordinary western blot methods, the APOBEC3G-fluorescent reporter assay, a) has higher throughput, b) requires less labor, c) is more quantitative, and d) has lower variability. The following chart compares the methods of the present invention with the prior art, showing how the methods of the present invention improve upon prior art methods.

| | | Fluorescent- |
|---|---|---|
| Experimental | Western Blot | Reporter |
| Step | Based Assays | Based Assay |
| Virus Preparation | No change | No change |
| TZM-bl Infection | No change | No change |
| Analysis of Cellular | SDS-PAGE followed by | Fluorescence |
| Apobec3G Levels | Western Blot | Measurement |
| Analysis of Virion | 3 Hr ultracentrifugation, | Fluorescence |
| Apobec3G Levels | SDS-PAGE, Western Blot | Measurement |

Ordinary assays to determine intracellular APOBEC3G involve manual lysis of cells by passing them ten times through a syringe followed by gel electrophoresis blot transfer and detection (two day process). Using the APOBEC3G-fluorescent reporter assay format described herein, a large number of samples can be completed in two hours.

Also, the regular assay to determine APOBEC3G incorporation into HIV virions requires two major steps and three days after harvesting of the virus containing cell supernatant (Virion pelleting and western blot). Using the APOBEC3G-fluorescent reporter assay format described herein, only one thirty-minute step is required.

In contrast to western blot format assays, the APOBEC3G-fluoresecent reporter assay is more robust, more reproducible, has lower variability.

The APOBEC3G-fluorescent reporter assay allows quantitative measurement of APOBEC3G protein incorporation into virions and APOBEC3G concentration in the virus producing cells. By normalizing the fluorescent reporter activity to a standard, the ability to compare compounds is improved over less accurate quantitation of prior art methods such as western blots. The quantitative ranking and comparisons of compounds by the degree of APOBEC3G stabilization is thus facilitated by the APOBEC3G-fluorescent reporter assays.

The APOBEC3G-fluorescent reporter assay allows for measurement of the potency of antiviral compounds that protect cellular APOBEC3G from degradation by HIV vif protein, leading to an increase of APOBEC3G incorporation in to HIV virions. The dynamic range of the APOBEC3G-luciferase assay between low APOBEC3G concentrations associated with wild-type HIV and high APOBEC3G concentrations associated with Δvif HIV is large. This allows for the robust assessment of antiviral potency of drug candidates and the generation of dose-response curves.

In all embodiments employing cotransfection of a wild-type or Δvif HIV genome together with an APOBEC3G-fluorescent reporter fusion, adjusting the relative ratio of plasmids encoding the wild-type (WT) or Δvif HIV genomes and A3G-luciferase (A3G-luc) for cotransfection in virus producer cells (e.g., 293T cells) is useful for achieving an optimal signal to background ratio and dynamic assay range for antiviral compound characterization. Adjusting the cotransfection ratio is also useful for obtaining a linear correlation between the fluorescent reporter (e.g., luciferase) signal and the level of A3G-fluorescent reporter protein detected by Western blotting. The luciferase activity ratio of (Δ vif HIV+A3G-luc) over (WT HIV+A3G-luc) in both virus supernatant and 293T cell lysates was measured. The results are presented in the following tables:

Experiment 1

| | RLU Ratio (delta vif HIV + | | | |
|---|---|---|---|---|
| | A3G-luc/WT HIV + A3G-luc) | | | |
| Co-transfection ratio | Virus sup. (RLU) | | 293T cell lysate (RLU) | |
| (WT HIV or delta vif | | 10 uM | | 10 uM |
| HIV: A3G-luc) | DMSO | MG132 | DMSO | MG132 |
| 19:1 | 2.8 | 3.2 | 5.0 | 2.6 |
| 39:1 | 1.1 | 5.5 | 4.5 | 2.4 |
| 79:1 | 20.3 | 5.9 | 6.0 | 3.7 |
| 159:1 | 12.3 | 4.7 | 4.8 | 6.8 |

Experiment 2

| | RLU ratio (delta vif HIV + | | | |
|---|---|---|---|---|
| | A3G-luc/WT HIV + A3G-luc) | | | |
| Co-transfection ratio | Virus sup. (RLU) | | 293 T cell lysate (RLU) | |
| (WT HIV or delta vif | | 10 uM | | 10 uM |
| HIV: A3G-luc) | DMSO | MG132 | DMSO | MG132 |
| 19:1 | 6.3 | 9.2 | 3.9 | 1.4 |
| 39:1 | 1.2 | 3.9 | 3.9 | 1.8 |
| 79:1 | 29.3 | 16.8 | 6.5 | 2.9 |
| 159:1 | 12.0 | 9.2 | 7.1 | 8.7 |

A plasmid cotransfection ratio of between about 50 to about 200 can be used in the assays of the invention for the detection of ≧10 fold change in the virus supernatant and ≧5 fold change in 293T cell lysates under conditions where there is no exposure to an inhibitory compound. A cotransfection ratio between about 50 and about 100 or between about 70 and about 90 is preferred to achieve consistent dynamic range for the assay. Alternatively, any plasmid cotransfection ratio that yields a ≧10 fold change in the virus supernatant and/or ≧5 fold change in 293T cell lysates under no compound conditions can be used.

### Biological Examples

### Example 1

### Cell-Based Screening Assay For Compounds That Inhibit Vif-Mediated APOBEC3G Degradation

Inhibitors of Vif-mediated APOBEC3G degradation were screened using a cell-based assay. A schematic of the assay is shown in FIG. 7A. The assay uses HeLa cells that stably expresses Vif and APOBEC3G fused at its C-terminus to firefly luciferase. Under steady-state conditions, there is very little luciferase activity. When degradation of the APOBEC3G-luciferase fusion is inhibited, a significant increase in the luciferase activity is observed. FIG. 7C shows the dose-response curve for the proteasome inhibitor MG132, which is known to increase APOBEC3G levels in the presence of Vif.

Construction of screening line (Vif/APOBEC3G-luciferase HeLa line). HeLa cells were cotransfected with a 1:1 ratio of pcDNA6-Vif and pcDNA3.1-APOBEC3G-luciferase plasmids using Fugene transfection reagent. Twenty-four hours after transfection, cells were trypsinized and different dilutions plated on 10 cm dishes. After attachment, stable transfectants were selected using medium containing 10 μg/ml blasticidin S. The blasticidin resistance gene is carried in the pcDNA6-Vif plasmid. Individual clones were plated in duplicate 96-well plates, treated with either DMSO or 10 μM MG132 for 24 hours, and then assayed for luciferase activity. A blasticidin-resistant clone with at least a ten-fold window in luciferase activity between DMSO and MG132 was grown out for HTS screening of the compound library. The presence of Vif and APOBEC3G-luciferase proteins was confirmed by treating cells with 10 μM MG132 overnight and analyzing cell lysates for both Vif and APOBEC3G-luciferase by western blotting (See FIG. 7B).

### Example 2

### Response of Vif/APOBEC3G-Luc Line To Proteasome Inhibitors

The screening assay of Example 1 was used to measure that inhibitory activity of proteasome inhibitors, such as small molecules known to increase APOBEC3G levels in the presence of Vif. The response of the screening line to different proteasome inhibitors show that compounds can be detected that targeted the degradation pathway. Compounds tested include MG-132, Velcade, Epoxomicin and Lactacystin.

### Example 3

### Diagram of HIV-Dependent Reporter Cell Line TZM-Bl

HIV infectivity was measured using the reporter cell line TZM-bl. TZM-bl is a HeLa line that contains two HIV-dependent reporter genes (Tranzyme). This line has been engineered to express the cell surface receptors CD4 and CCR5 as well as two reporter genes whose expression is driven by an HIV specific promoter (the cell has to be successfully infected with HIV in order to get expression of the reporter genes firefly luciferase and β-galactosidase). Because this line expresses CD4 and CCR5 (HeLa cells naturally express CXCR4, the co-receptor for X4-tropic viruses), it can be utilized for infectivity analyses of any HIV-1, HIV-2, or SIV strain. This line is also exquisitely sensitive—luciferase activity can be detected from as few as 30 infected cells in a population.

### Example 4

### Measuring Infectivity of Virus Particles Generated in the Presence of Different Compounds

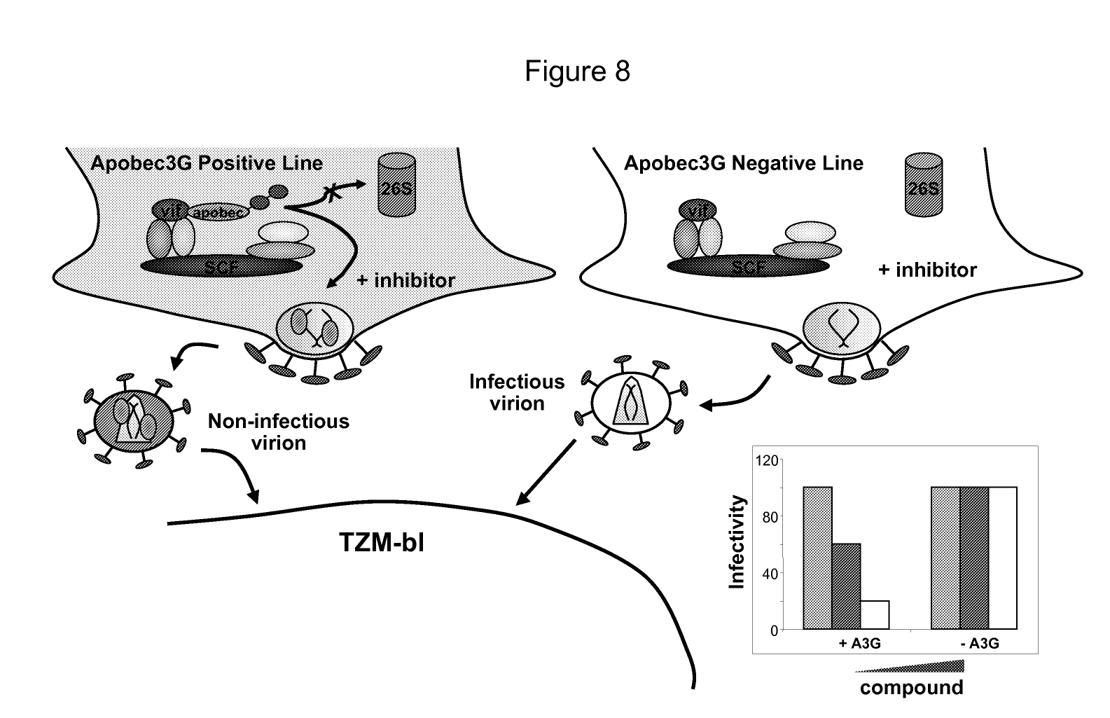

The effect of candidate inhibitor compounds on infectivity was measured in a single-cycle infectivity measurement assay, as opposed to measuring compound efficacies in a spreading infection experiment. FIG. 8 shows a schematic of the single-cycle infectivity assay. The single-cycle measurement is faster than the spreading infection and has higher throughput. An important aspect of this assay is that virus particles are generated in both cells that express APOBEC3G and in cells that lack APOBEC3G. After collecting the viral supernatants, the amount of capsid (p24) protein in the supernatant was measured and used to standardize the amount of input virus to infect the TZM-bl cells.

Generation of viruses. 293T cells were transfected in 6 well dishes with a 9:1 ratio of either HIV expression plasmid and pcDNA3.1-APOBEC3G or HIV expression plasmid and pcDNA3.1 (empty vector) using Fugene. On the day following transfection, cells were pooled and then replated. The following day, the medium was aspirated and replaced with medium containing indicated compound concentrations. Twenty-four hours after initiation of compound treatment, virus supernatants were collected and filtered through a 0.45μ filter. Viral supernatants were divided into aliquots and frozen at −80° C. Transfected cells were lysed using SDS-PAGE loading buffer for analysis of cellular and viral proteins by western blotting. Alternatively, viruses can be generated using T lymphocyte cells as virus producing cells. T cells (either H9 or CEMSS-A3G) were infected by mixing 6×10⁶ target cells with 100 ng p24 in 2 mls of medium and then spinning the sample at 1000 g for one hour. After spinning, the samples were transferred to a 37° C. incubator. After 6 hours at 37° C., the samples were brought to a density of 1×10⁶/ml. Cells were maintained at 1×10⁶/ml and monitored for HIV infection by sampling aliquots for intracellular HIV gag staining using flow cytometry. When the cell population reaches 30% infection, cells were pelleted and then resuspended in fresh medium at a concentration of 1×10⁶ cells/ml. Compounds were added at appropriate concentrations (6×10⁶ cells were used per sample). After 20 hours, viruses were harvested as above.

Infection of HeLa TZM-bl indicator lines. Virus concentrations in cell supernatants were quantitated using p24 ELISA. One day before infection, 1×10⁵ TZM-bl cells per well were plated in 24 well dishes. Virus stocks were diluted to 5 ng p24/ml and then added to each well of TZM-bl cells. The plates were incubated at 37° C. for 6 hours, washed once with PBS, and then fresh medium added. Thirty hours after initiation of infection, cells were washed with PBS, and then lysed using 100 μL Bright-Glo lysis buffer per well. Twenty microliters of each infection was assayed in duplicate for luciferase activity using Bright-Glo firefly luciferase substrate.

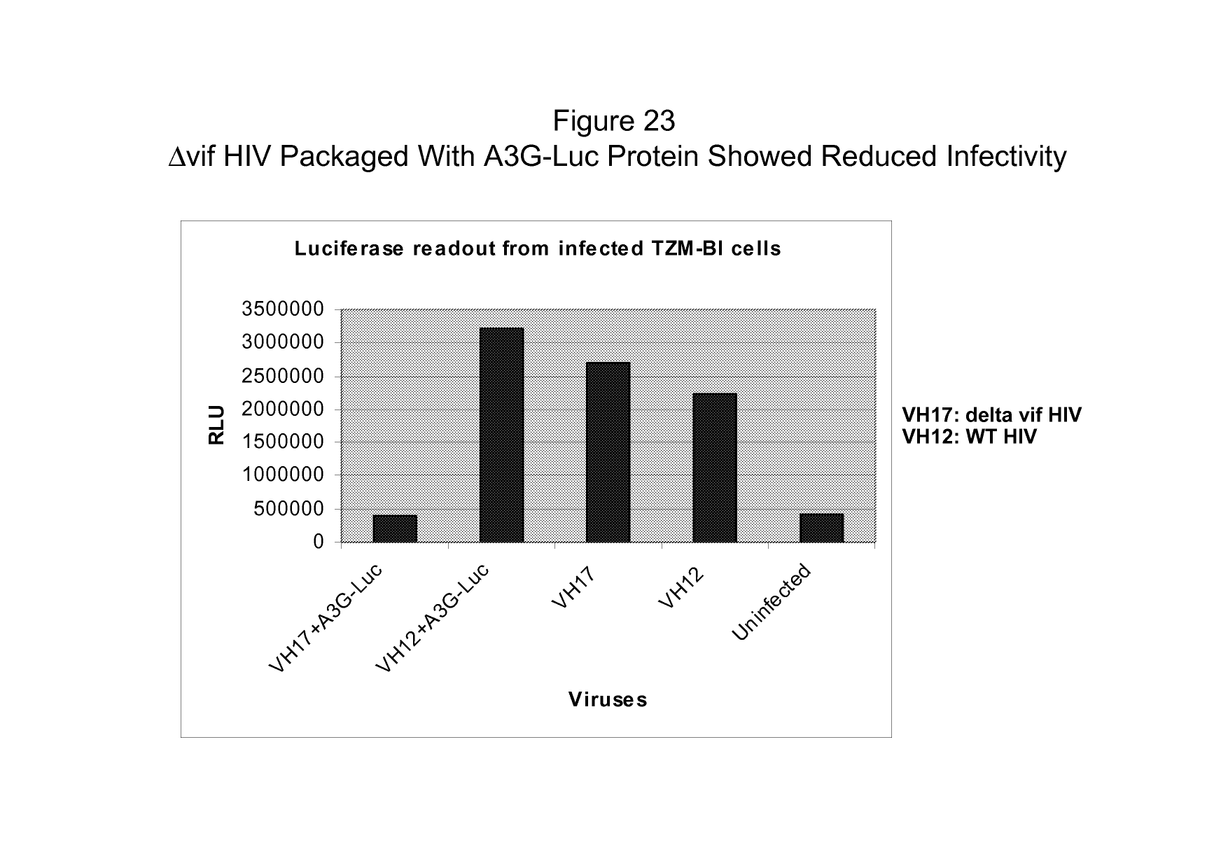

FIG. 23 shows the luciferase readout for infected TZM-bl indicator lines. There is reduced infectivity and a lower luciferase readout for HeLa TZM-bl cells expressing APOBEC3G when infected by HIV without the vif gene.

### Example 5

### Determination of a Compound's Antiviral Activity in the Virus Producing Cells using Luciferase Activity

293T cells were seeded in 6-well plates at 500,000 cells/well density. On the second day, 4 μg of wild-type or delta vif HIV-1 construct and pcDNA3.1-APOBEC3G-luciferase construct plasmid DNA were co-transfected into 293T cells using Lipofectamine 2000 (Invitrogen). On the third day, cells were detached with trypsin and plated into 24-well plates at 300,000 cells/well density. On the fourth day, medium was replaced in each well with either 0.5% DMSP medium or with compound and 0.5% DMSO containing medium.

Twenty hours post dosing, the virus producing 293T cells of Example 5 were lysed with 200 μL of Bright-Glo lysis buffer in 24-well plates. Cell lysates were diluted 1:100 in PBS. 10 μL of each lysate sample were transferred to small well 96-well plates and 10 μL of Bright-Glo substrate were added to each well. The luciferase activity from the cell lysates was then detected using a Top Count reader. The total protein concentration was determined using the Quant-IT Protein Assay Kit (Molecular Probes). The luciferase activity was normalized relative to total cellular protein by dividing the background corrected luciferase light units by the total protein concentration.

### Example 6

### Determination of a Compound's Antiviral Activity in Virus Supernatant Using Luciferase Activity

Twenty hours post dosing, the culture supernatant from Example 5 was harvested and filtered through a 0.45μ filter. Twenty microliters of virus supernatant from each sample were transferred into a 96-Well plate. 30 μL of culture medium and 50 μL of Bright-Glo lysis buffer (Promega), were added to each well, followed by 50 μL of Bright-Glo Substrate (Promega). The luciferase activity from the virus supernatant was then detected using a Top Count reader (Perkin Elmer). The concentration of virus particles in the supernatant was determined by p24 ELISA (Perkin Elmer). The luciferase activity of the virus supernatant was then normalized relative to the concentration of virus particles by dividing the background corrected luciferase light units by the p24 values.

### Example 7

### Determination Of A Compound's Antiviral Activity Due To APOBEC3G Function

This assay is used for determining whether a compound's antiviral activity is due to APOBEC3G function as opposed to general antiviral activity of unknown origin. Several different measurements from the single-cycle infectivity assay can be obtained. From western blots on lysates from the 293T virus producer cells, information about whether or not the compounds increase APOBEC3G levels in a dose-dependent fashion can be extracted. By infecting TZM-bl cells with the viral supernatants and measuring the resulting HIV-dependent luciferase activity, measurement of the infectivity of the virus particles is obtained. By comparing the infectivity of the particles generated in cells that express APOBEC3G to the infectivity of the particles from cells that lack APOBEC3G, a determination of whether the antiviral activity is due to APOBEC3G function can be derived. This is because in cells expressing APOBEC3G that are treated with compound, an increase of APOBEC3G levels is expected which results in virion incorporation of an antiviral factor, decreasing the infectivity of those virions. In cells without APOBEC3G but treated with compound, no antiviral factor gets packaged into virions so these virions should retain their infectivity. Under these conditions, a dose-response data like that shown in FIG. 8 would indicate that virion infectivity would decrease with increasing compound concentration in the presence of APOBEC3G and no dose-dependent effect in the absence of APOBEC3G.

### Example 8

### Identification of Inhibitors of APOBEC3G Degradation

Using the methods of Examples 1-7 above, several candidate compounds were tested and identified as inhibitors of APOBEC3G degradation and HIV infectivity.

### Example 9

### Protocol For Virion Incorporation

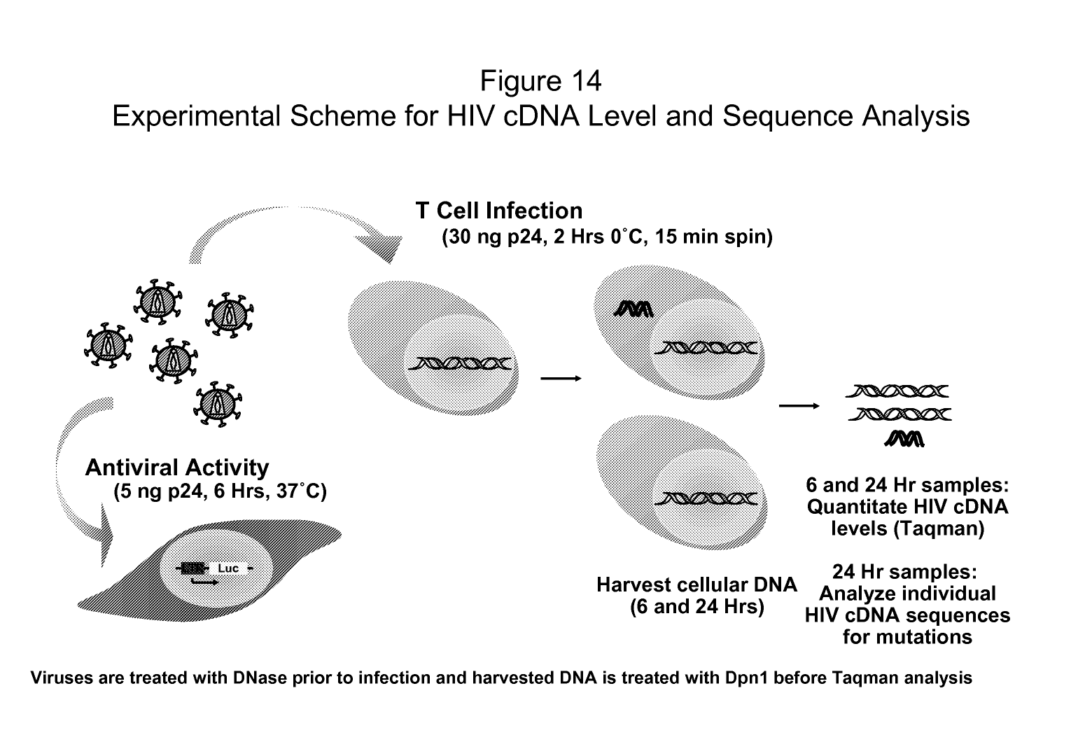

The data shown in FIGS. 14-16 and 24 were obtained using the same virus stocks for the different experiments. Infections performed with ΔVif virus are used as a control for each experiment.

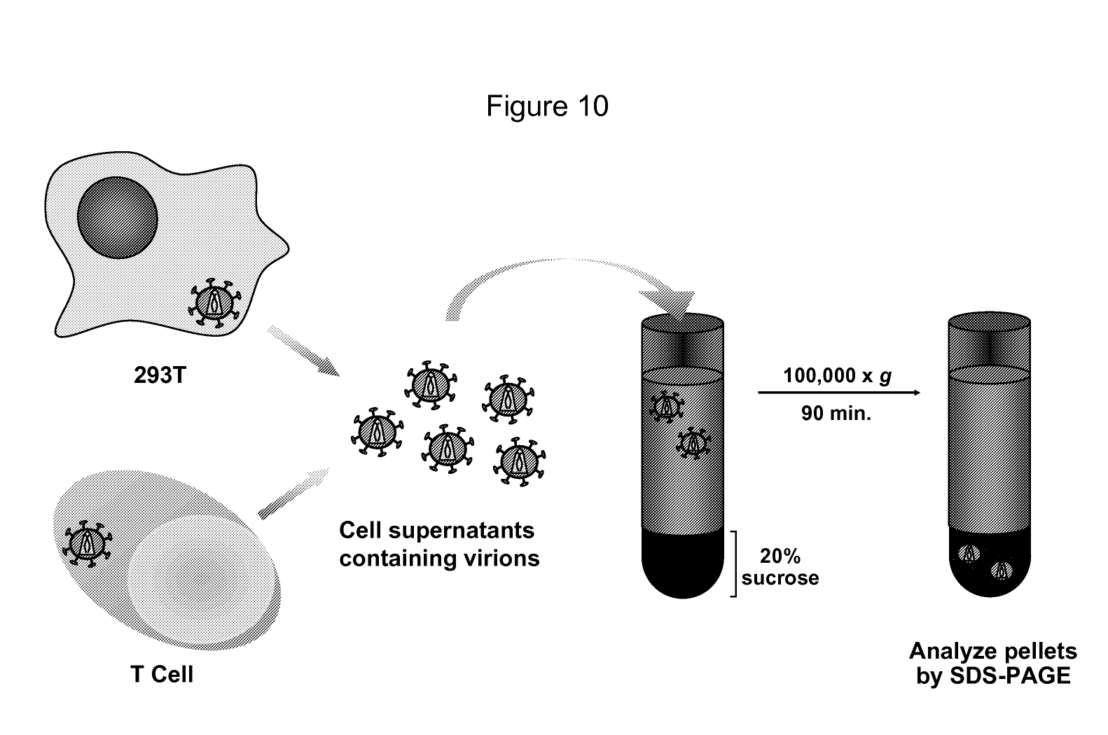

Isolation of virions using sucrose sedimentation (FIG. 10). Viral supernatants (3 mls) were added carefully to 5.5 mls of medium overlaid onto a 2 ml 20% sucrose/PBS cushion in a 14 ml ultracentrifuge tube. Samples were spun using an SW41 rotor for 90 minutes at 41,000 rpm at 4° C. The medium and the sucrose cushion were carefully removed by aspiration and then 50 μL of SDS-PAGE loading buffer was used to resuspend the pelleted virions. Ten microliters of each sample was used for western blotting analysis.

FIG. 20 shows a dose-dependent virion incorporation of APOBEC3G. FIG. 20A is the virion infectivity measured using TZM-bl line and assay of Example 3. FIG. 20B is a western blot of APOBEC3G levels in virus producer cells. Levels of lactate dehydrogenase (LDH) are shown as a cellular loading control. FIG. 20C is a western blot of APOBEC3G levels in purified virions. Levels of capsid protein (p24) are shown as a loading control.

### Example 10

### Reduction of HIV Reverse Transcripts

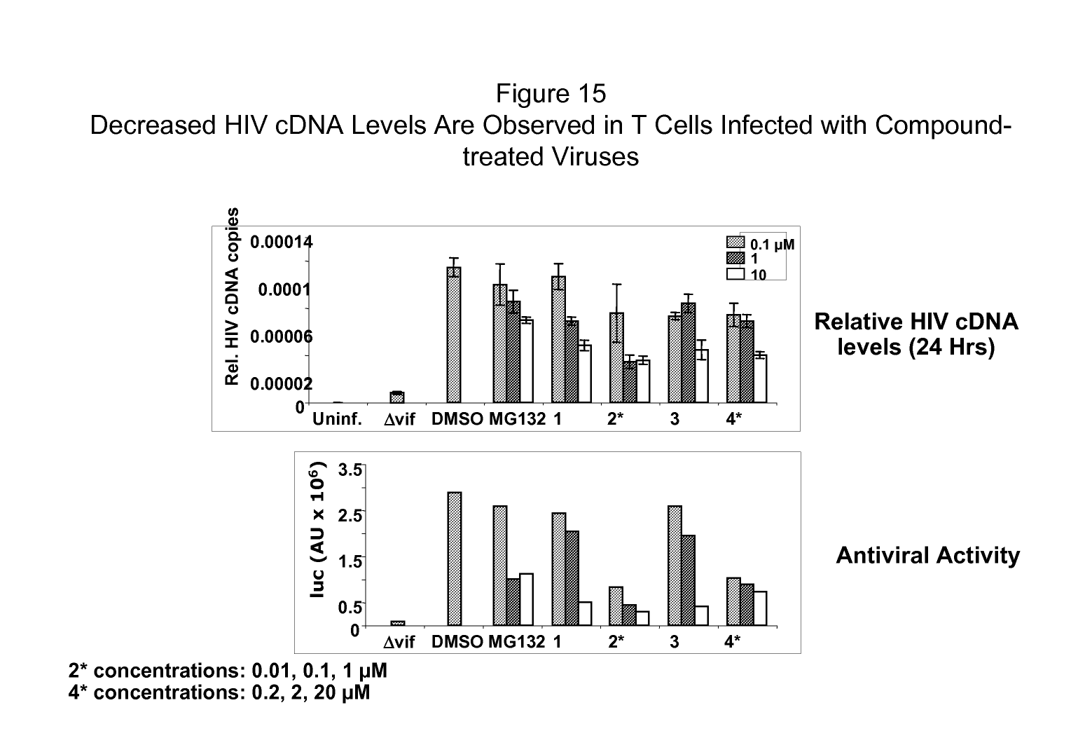

FIG. 15 shows the dose-dependent reduction in HIV reverse transcripts. Quantitative PCR Analysis of HIV reverse transcripts were performed as follows. Prior to infection, virions were treated with Rnase-free Dnase (10 μL/500 μL virus) at room temperature for one hour to remove input transfected DNA. Dnase-treated viral supernatants were diluted to 15 ng p24/ml. Two mls of diluted virus was mixed with 1×10⁶ SupT1 cells and incubated on ice for 2 hours. Samples were transferred to 6 well plates and spun at room temperature in a benchtop centrifuge for 15 minutes at 2500 rpm. Cells were washed once with PBS and then mixed with two mls of complete medium and placed at 37° C. Duplicate samples were prepared so that one set could be harvested six hours after the start of infection and the other twenty-four hours after the start of infection. Total DNA was isolated using the DNeasy kit from Qiagen and eluted in a volume of 200 μL. DNA was treated with Dpn1 for 2 hours at 37° C. and then 2 μL of each sample was used for real time quantitative PCR analysis. Reverse transcripts were detected using primers that amplify the region between 500 and 695 of the provirus: 500F (5′-TAACTAGGGAACCCACTGC-3′), 695R (5′-CTGCGTCGAGAGAGCTCCTCTGGTT-3′), and RT probe (5′-FAM-ACACAACAGACGGGCACACACTA-TAMRA-3′). Reactions were performed in triplicate, in Taqman Universal PCR master mix, using 0.9 μmol of each primer/μL and 0.25 μmol of probe/μL in a total volume of 50 μL. PCR cycling conditions used were: 10 minutes at 95° C., followed by 40 cycles of 15 s at 95° C., 1 minute at 60° C. Serial dilutions of the HIV proviral vector pIIIb were used as control samples to confirm the linearity of the assay and also used for relative quantitation of the samples. The phrase “compound-treated virus” as used in FIG. 15 and throughout the specification and claims refers to virus produced in cells that are treated with a compound. A schematic for the determining cDNA levels and sequencing is shown in FIG. 14.

### Example 11

### Increased Mutations in HIV cDNAs

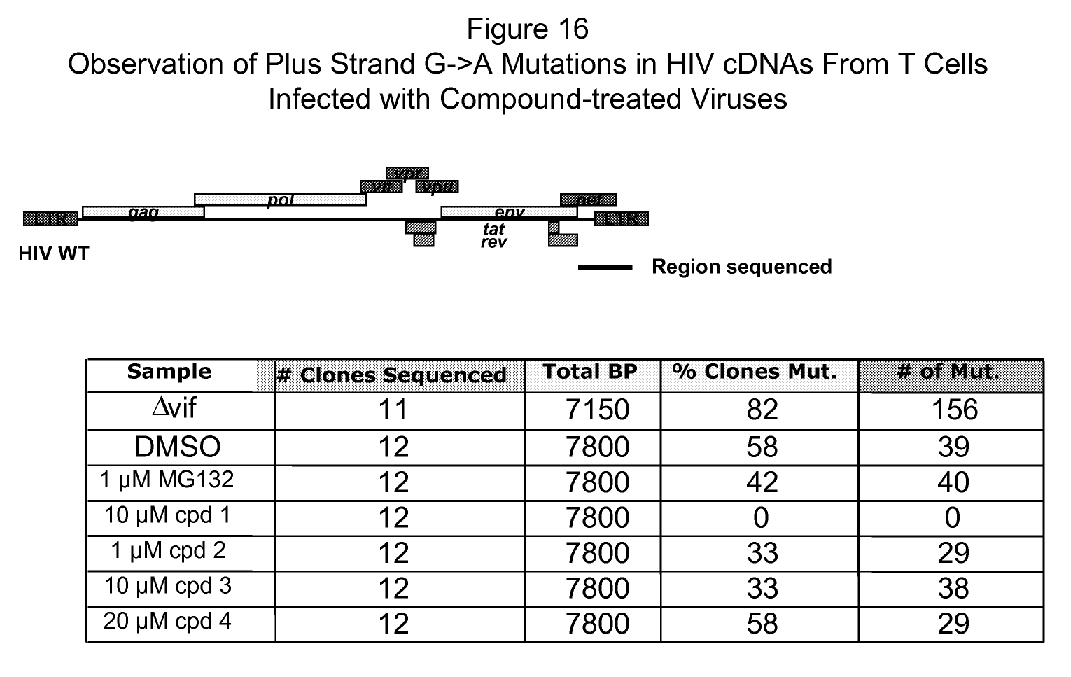

FIG. 16 shows the sequence analysis of HIV cDNAs. Mutations in the DMSO sample were observed because the viruses are produced in the presence of APOBEC3G. Vif clearly reduces the effect of APOBEC3G (compare ΔVif versus DMSO) but APOBEC3G is not entirely eliminated from the virion. What can be concluded from this data is that, for all of the compounds except for one, enzymatically active APOBEC3G is present in the virion.

Sequencing analysis of HIV cDNAs. A 650 bp region extending from nef to the U5 region of the 3 ′UTR (untranslated region) was amplified from total DNA samples prepared for the real time quantitative PCR analysis using the HIV-1 specific primers Nef.s.(5′-CCGAATTCAGGCAGCTGTAGATCTTAGCCACTT-3′) and U5.a. (5′-CAGGATCCGGTCTGAGGGATCTCTAGTTAC-3′) and Advantage HF-2 polymerase (BD Biosciences). The PCR products were gel-purified, digested with EcoRI and BamHI, and ligated into pBluescript using the EcoRI and BamHI cloning sites. All pBluescript clones were sequenced using T7 forward and M13 reverse sequencing primers. Bishop et al. (J. Virol. (2006) 8450-8458) further discusses the quantitation of transcripts and cDNAs sequencing, and is incorporated by reference in its entirety.

### Example 12

### Compound Effect on Viral Replication in T Cells Expressing APOBEC3G (H9,MT2) Versus T Cells that Lack APOBEC3G Expression (CEMSS-PURO)

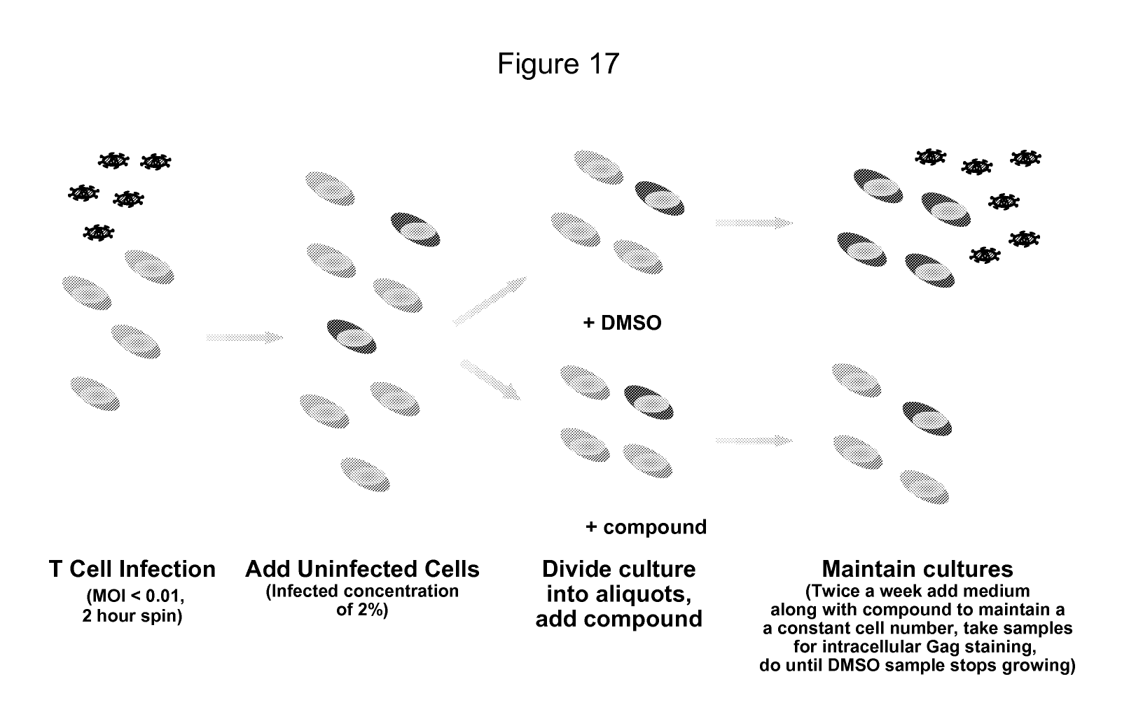

About 1.2×10⁶ T cells were spin-infected with 2 ml of virus at an MOI of 0.01 for 2 hours at room temperature. Infected cells were washed once with PBS and then added to uninfected cells such that the final concentration of infected cells was 2%. Cells were evenly aliquoted into 17 samples and either DMSO or compound added to each sample. Every other day, samples were counted and fresh medium added to maintain the cells at a concentration of 5×10⁵/ml. In addition, fresh compound was added to maintain a constant compound concentration in the samples. All samples were maintained until cells in the DMSO control stopped growing. The extent of virus replication in the cultures was measured by analyzing the intracellular HIV gag staining by flow cytometry at each time point. FIG. 17 shows a schematic of this experiment.

What is claimed is:

1. A method of identifying inhibitors of HIV infectivity comprising:
a) producing HIV virus particles in two cultures of virus producing cells, wherein the HIV virus particles are produced in the first culture in the presence of a test compound and in the second culture in the absence of the test compound;
b) measuring the amount of APOBEC3G in the HIV virus particles produced by the virus producing cells; and
c) comparing the amount of APOBEC3G in the HIV virus particles produced in the virus producing cells in the presence of the test compound to the amount of APOBEC3G in HIV virus particles produced in the virus producing cells in the absence of the test compound,
wherein a greater amount of APOBEC3G in the HIV virus particles produced from the virus producing cells in the presence of the test compound indicates that the test compound is an inhibitor of HIV infectivity.

2. A method of identifying an inhibitor of HIV infectivity comprising:
a) producing HIV virus particles in a first cell culture and a second cell culture, wherein the HIV virus particles are produced in the first cell culture in the presence of a test compound and in the second cell culture in the absence of the test compound;
b) infecting a third and fourth cell cultures with the HIV virus particles produced in the first and second cell cultures, respectively;
c) culturing the third and fourth cell cultures;
d) measuring the level of HIV nucleic acid in the third and fourth cell cultures; and
e) comparing the amount of HIV nucleic acid in the third cell culture to the amount of HIV nucleic acid in the fourth cell culture, wherein a lesser amount of HIV nucleic acid in the third cell culture compared to the fourth cell culture indicates that the test compound is an inhibitor of HIV infectivity.

3. A method of identifying an inhibitor of HIV infectivity comprising:
a) producing HIV virus particles in a first cell culture and second cell culture, wherein the HIV virus particles are produced in the first cell culture in the presence of a test compound and in the second test culture in the absence of the test compound;
b) infecting a third cell culture and a fourth cell culture with the virus produced in the first cell culture and second cell culture, respectively;
c) culturing the third and fourth cell cultures;
d) measuring the amount of mutations in HIV nucleic acid in the third and fourth cell cultures; and
e) comparing the amount of mutations in HIV nucleic acid in the third cell culture to the amount of mutations in HIV nucleic acid the fourth cell culture,
wherein a greater amount of mutations in HIV nucleic acid in the third cell culture compared to the fourth cell culture indicates that the test compound is an inhibitor of HIV infectivity.

4. A method of identifying an inhibitor of vif-mediated APOBEC3G degradation, comprising:
a) producing HIV virus particles in first and second virus producing cell cultures, wherein the HIV virus particles are produced in the first cell culture in the presence of a test compound and in the second test culture in the absence of the test culture, wherein the virus producing cells of the first and second cell cultures contain APOBEC3G;
b) producing HIV virus particles in third and fourth virus producing cell cultures, wherein the HIV virus particles are produced in the third cell culture in the presence of a test compound and in the fourth cell culture in the absence of the test compound, wherein the third and fourth virus producing cell cultures do not contain APOBEC3G;
c) measuring the infectivity of the HIV virus particles produced in the first, second, third, and fourth virus producing cell cultures;
d) comparing the infectivity of the HIV virus particles produced in the first cell in the first cell culture to the infectivity of the HIV virus particles produced in the second cell culture; and
e) comparing the infectivity of the HIV virus particles produced in the third cell culture to the infectivity of the HIV virus particles produced in the fourth cell culture,
wherein a reduction of infectivity of the HIV virus particles produced in the first cell culture compared to the second cell culture and no reduction in infectivity of the HIV virus particles produced in the third cell culture compared to the fourth cell culture indicates that the test compound is an inhibitor of vif-mediated APOBEC3G degradation.

5. A method of identifying an inhibitor of vif-mediated APOBEC3G degradation comprising:
a) providing a cell that expresses Vif and an APOBEC3G fusion protein, wherein the APOBEC3G fusion protein comprises APOBEC3G fused to a reporter molecule;
b) contacting the cells with a compound to be tested under conditions that allow entry of the compound into the cell; and
c) detecting a signal of the reporter molecule;
wherein the magnitude of the signal is proportional to the inhibitory activity of the compound.

6. The method of claim 5, wherein the cells are HeLa cells.

7. The method of claim 5, wherein the reporter molecule is luciferase.

8. The method of claim 5, wherein the cells are T-lymphocyte cells.

9. The method of claim 7, wherein the signal of the reporter molecule is measured in growth supernatant of the cells.

10. The method of claim 7, wherein the signal of the reporter molecule is measured in lysate of the cells.

11. A method of assaying APOBEC3G derived from HIV comprising
a) providing a cell comprising at least one reporter gene, wherein expression of the at least one reporter gene is induced by an HIV specific promoter, wherein the cell comprises at least one type of HIV receptor;
b) contacting the cell with HIV under conditions that allow entry of HIV into the cell; and
c) measuring a signal generated by the reporter gene;
wherein the magnitude of the signal is inversely proportional to the amount of APOBEC3G in the HIV.

12. The method of claim 11, wherein the cell is HeLa line.

13. The method of claim 11, wherein the cell comprises two reporter genes.

14. The method of claim 13, wherein the reporter genes are genes for luciferase and β-galactosidase.

15. The method of claim 11, wherein the cell comprises HIV receptors CD4 and CCR5.

16. A method of identifying inhibitors of APOBEC3G degradation comprising
a) providing a first cell, wherein the first cell is an HIV producer cell;
b) co-transfecting the first cell with HIV and APOBEC3G;
c) contacting the transfected first cell with a compound to be tested under conditions that allow entry of the compound into the cell;
d) harvesting HIV produced by the first cell after contacting the first cell with the compound;
e) providing a second cell comprising at least one reporter gene, wherein expression of the at least one reporter gene is driven by an HIV specific promoter, wherein the second cell comprises at least one type of HIV receptor;
f) contacting the second cell with HIV harvested from d) under conditions that allow entry of HIV into the cell; and
g) measuring a signal from the reporter gene;
wherein the magnitude of the signal is inversely proportional to the inhibitory activity of the compound.

17. The method of claim 16, wherein the first cell is a 293T cell and second cell is HeLa cell.

18. The method of claim 16, wherein the first cell is a T-lymphocyte cell and second cell is HeLa cell.

19. The method of claim 16, wherein the first cell is H9 cell and second cell is HeLa cell.

20. The method of claim 16, wherein the FIRST cell is CEMSS-A3G cell and second cell is HeLa cell.

21. The method of claim 16, wherein the second cell comprises two reporter genes.

22. The method of claim 21, wherein the reporter genes are genes for luciferase and β-galactosidase.

23. The method of claim 16, wherein the second cell comprises HIV receptors CD4 and CCR5.

24. A cell comprising genes that code for Vif and APOBEC3G fusion protein, wherein the gene that codes for APOBEC3G fusion protein comprises a reporter gene that codes for a molecule that possesses or is capable of generating or inducing, directly or indirectly, a detectable signal.

25. The cell of claim 24, wherein the reporter gene codes for firefly luciferase.

26. The cell of claim 24, wherein the product of the gene that codes for APOBEC3G fusion protein is an APOBEC3G protein fused at the C-terminus to firefly luciferase.

27. The cell of claim 24, wherein the cell is a 293T cell.

28. The cell of claim 24, wherein the cell is a T-lymphocyte cell.

29. The cell of claim 24, wherein the cell is an H9 cell.

30. The cell of claim 24, wherein the cell is a CEMSS-3AG cell.


SEQUENCE LISTING (5 sequences)

SEQ ID NO: 1 - DNA, 19 bp, Artificial
  Synthetic; Primer for HIV reverse transcriptase.
taactagggaacccactgc

SEQ ID NO: 2 - DNA, 25 bp, Artificial
  Synthetic; Primer for HIV Reverse Transcriptase
ctgcgtcgagagagctcctctggtt

SEQ ID NO: 3 - DNA, 23 bp, Artificial
  Synthetic; HIV Reverse Transcriptase Probe
acacaacagacgggcacacacta

SEQ ID NO: 4 - DNA, 33 bp, Artificial
  Synthetic; HIV-1 Specific Primier
ccgaattcaggcagctgtagatcttagccactt

SEQ ID NO: 5 - DNA, 30 bp, Artificial
  Synthetic; HIV-1 Specific Primer
caggatccggtctgagggatctctagttac